(19) United States
(12) Patent Application Publication
(10) Pub. No.: US 2025/0152676 A1
(43) Pub. Date: May 15, 2025

(54) METHOD AND PHARMACEUTICAL COMPOSITION FOR TREATING CHRONIC OBSTRUCTIVE PULMONARY DISEASE

(71) Applicant: Jiu-Yao Wang, Tainan City (TW)

(72) Inventors: Jiu-Yao WANG, Tainan City (TW); Miao-Hsi HSIEH, Changhua County (TW)

(73) Assignee: Jiu-Yao WANG, Tainan City (TW)

(21) Appl. No.: 18/389,544

(22) Filed: Nov. 14, 2023

(51) Int. Cl. A61K 38/17; A61P 11/16

(52) U.S. Cl. CPC A61K 38/395; A61P 11/16

(57) ABSTRACT

The present invention provides a new therapy of chronic obstructive pulmonary disease (COPD) using surfactant protein-D (SP-D) or its fragment.

### FIELD OF THE INVENTION

The present invention relates to a new method and pharmaceutical composition for treating chronic obstructive pulmonary disease.

### REFERENCE TO ELECTRONIC SEQUENCE LISTING

The application contains a Sequence Listing which has been submitted electronically in .XML format and is hereby incorporated by reference in its entirety. Said .XML copy, created on Nov. 13, 2023, is named “2024-01-29-SeqListing-5992-0472PUS1” and is 12,083 bytes in size. The sequence listing contained in this .XML file is part of the specification and is hereby incorporated by reference herein in its entirety.

### BACKGROUND OF THE INVENTION

Chronic obstructive pulmonary disease (COPD) is a serious lung disease that has exhibited increasing morbidity and mortality worldwide [1]. It is the seventh leading cause of death in Taiwan [2] and third in the United States [3]. Although the underlying pathogenic mechanism of COPD remains unclear, inhaled oxidants such as cigarette smoke (CS) and air pollution dysregulate the expression of proteolytic enzymes and reactive oxygen species (ROS) that contribute to the production of inflammatory mediators and the release of metalloproteases from alveolar macrophages (AMs) to damage alveolar structure [4]. Existing therapeutic approaches are largely ineffective against the progressive deterioration of lung function and mortality in COPD [1].

Accordingly, it is still desirable to develop a new approach to treat COPD.

### BRIEF SUMMARY OF THE INVENTION

Accordingly, the present invention provides a new approach for treating Chronic obstructive pulmonary disease (COPD).

In one aspect, the present invention provides a method for treating Chronic obstructive pulmonary disease (COPD) in a subject, comprising administering to a subject an therapeutically effective amount of surfactant protein-D (SP-D).

In another aspect, the present invention provides a composition or pharmaceutical composition for treating COPD comprising a therapeutically effective amount of SP-D, and a pharmaceutically acceptable carrier.

In a further aspect, the present invention provides a use of SP-D for manufacturing a medicament for treating COPD.

In one embodiment of the invention, the SP-D is a native SP-D or a fragment or a recombinant fragment thereof, e.g., a recombinant fragment of human SP-D (rfhSP-D).

In one particular example of the invention, the native SP-D is the SP-D consisting of the amino acid sequence of SEQ ID NO:1.

In one particular example of the invention, the rfhSP-D is the fragment consisting of the amino acid sequence of SEQ ID NO:2.

In the invention, it was ascertained that using a mouse model of COPD, exogenous administration of a native SP-D or a rfhSP-D prevented the formation of lipid-laden foamy macrophages (FMs) by regulating lipid metabolism, thereby protecting against airway inflammation and alveolar emphysematous changes.

It is to be understood that the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The drawings presenting the preferred embodiments of the present invention are aimed at explaining the present invention. It should be understood that the present invention is not limited to the preferred embodiments shown.

FIGS. 1A-1F illustrate that ozone induced lung inflammation and increased the level of SP-D in serum and BALF:

FIG. 1A shows that the BALB/c female mice were exposed to 1 and 3 ppm ozone for 3 h 3 times per week; after exposure, lung function was determined using Snapshot150 perturbation on a FlexiVent (SCIREQ) (the data represent the mean±SEM. *p<0.05, **p<0.01, ***p<0.001, two-way ANOVA vs. the air group);

FIG. 1B shows H&E staining of lung sections (scale bar=100 μm);

FIG. 1C shows that the alveolar well length was measured by the MLI;

FIG. 1D shows that BALF cell counts included total cells, macrophages (macro), neutrophils (neu), basophils (baso), and lymphocytes (lymph) (the data represent the mean±SEM. **p≤0.01; ***p≤0.001, Student's unpaired t test);

FIG. 1E shows that the levels of TNF-α, IL-6 and CXCL1 in the BALF were measured by ELISA (the data represent the mean±SEM. *p<0.05, **p≤0.01, ***p≤0.001, Student's unpaired t test);

FIG. 1F shows that the levels of SP-D in serum were measured by ELISA (the data represent the mean±SEM. *p<0.05, **p≤0.01, ***p<0.001, Student's unpaired t test).

FIGS. 2A-2E illustrate that CS induced lung inflammation and increased the level of SP-D in serum and BALF:

FIG. 2A provides the protocol of a mouse model of CS-induced COPD used, wherein the mouse were exposed to the smoke of 6 burning cigarettes for 1 hour, 5 times per week for 6 weeks;

FIG. 2B shows that the CS exposure significantly increased the Rrs, Ers, and G after 200 mg/mL methacholine challenge compared to those in control mice exposed to air (the data represent the mean±SEM. *p<0.05, **p<0.01, ***p<0.001, two-way ANOVA vs. the air group);

FIG. 2C provides the images of H&E staining showing FCS exposure increased mucus production in the bronchioles and caused emphysema (scale bar=100 μm);

FIG. 2D shows the expression of cytokines and chemokines (TNF-α, IL-6, and CXCL1) after CS exposure;

FIG. 2E shows the level of SP-D in the serum and BALF after CS exposure.

FIGS. 3A-3F show that the depletion of SP-D exacerbated lung inflammation in ozone-exposed mice:

FIG. 3A shows that in the ozone-exposed C57BL/6J female mice, the levels of SPD in serum and BALF were measured by ELISA (the data represent the mean±SEM. *p<0.05, **p<0.01, ***p<0.001, two-way ANOVA vs. the air group);

FIG. 3B shows that the lung function was determined using Snapshot150 perturbation on FlexiVent (SCIREQ) (the data represent the mean±SEM. *p<0.05, **p<0.01, ***p<0.001, two-way ANOVA vs. the air group);

FIG. 3C shows that the alveolar well length was measured by the MLI;

FIG. 3D provides an image of the H&E staining of lung sections;

FIG. 3E provides the BALF cell counts including total cells, macrophages (macro), neutrophils (neu), basophils (baso), and lymphocytes (lymph) (the data represent the mean±SEM. **p≤0.01; ***p≤0.001, Student's unpaired t test);

FIG. 3F shows that the cells in BALF were stained with Liu and observed by light microscopy (scale bar=100 μm)

FIG. 3G shows that the levels of TNF-α, IL-6, and CXCL1 were measured by ELISA (the data represent the mean±SEM. *p<0.05, **p≤0.01, ***p≤0.001, Student's unpaired t test).

FIGS. 4A-4F illustrate that exogenous SP-D alleviated lung function and inflammation and reduced cell infiltration:

FIG. 4A shows that the ozone-exposed BALB/c female mice were treated with SP-D or PBS once per week, and lung function was determined using Snapshot150 perturbation on a FlexiVent (SCIREQ) (the data represent the mean±SEM. *p<0.05, **p<0.01, ***p<0.001, two-way ANOVA vs. the air group);

FIG. 4B provides an image of the H&E staining of lung sections (scale bar=100 μm);

FIG. 4C provides the alveolar well length measured by the MLI (the data represent the mean±SEM. **p≤0.01; ***p≤0.001, Student's unpaired t test);

FIG. 4D provides the BALF cell counts including total cells, macrophages (macro), neutrophils (neu), basophils (baso), and lymphocytes (lymph) (the data represent the mean±SEM. **p≤0.01; ***p≤0.001, Student's unpaired t test);

FIG. 4E shows the levels of SP-D in serum and BALF measured by ELISA (the data represent the mean±SEM. *p<0.05, **p≤0.01, ***p≤0.001, Student's unpaired t test);

FIG. 4F shows the levels of TNF-α, IL-6, and CXCL1 measured by ELISA (the data represent the mean±SEM. *p<0.05, **p≤0.01, ***p≤0.001, Student's unpaired t test).

FIGS. 5A-5J show that the SP-D pretreatment decreased ROS production and early-stage apoptotic cells and induced the expression of Nrf2:

FIG. 5A provides the bioluminescence imaging of ROS production in the lungs of air-, ozone-, and cigarette smoke-exposed COPD C57BL/6J female mice performed with an L-012 luminescent probe (IVIS Lumina LT Series III, PerkinElmer, USA);

FIG. 5B shows the levels of ROS production as examined by DCFDA in A549, BES-2B cells, and BMDMs which were pretreated with SP-D followed by H₂O₂;

FIG. 5C shows the early-stage apoptosis (annexin V+ and PI−) in A549 and BES-2B cells measured by flow cytometry;

FIG. 5D shows the percentage of cells in early apoptosis (A549 and BEAS-2B);

FIG. 5E provides the Nrf2 and β-actin protein expression in the A549 cells pretreated with 1, 2, 5, or 10 μg/ml SP-D and then treated with H₂O₂, respectively, as examined by western blotting, showing that the SP-D pretreatment yielded a dose-dependent increase in Nrf2 expression;

FIG. 5F shows the Nrf2 and β-actin expression in the lung tissues of wild-type as examined by western blotting;

FIG. 5G shows the Nrf2 expression in the SP-D−/− mice as examined by western blotting;

FIG. 5H shows the Nrf2 expression in the lung tissue after SP-D administration, as examined by IHC (scale bar=100 μm; wherein BMDMs were treated with medium (m), recombinant full length-SP-D (SP-D), and cigarette extract (CS) or were pretreated with SP-D and then treated with cigarette extract (SP-D+CS);

FIG. 5I shows that in a two-fold-change analysis, SP-D+CS regulated the expression of 21 genes compared with CS;

FIG. 5J shows the gene heatmap of log 2-transformed ratios of oxidative stress gene expression; wherein the left gene heatmap shows the log 2 ratios of CS, SP-D+CS, and SP-D treatment normalized to the medium control (CS/m, SP-D+CS/m, and SP-D/m); and the right gene heatmap shows the log 2 ratios of SP-D+CS normalized to CS (SP-D+CS/CS).

FIGS. 6A-6C provide the heatmaps showing the oxidative stress-related genes that are regulated by SPD performed by RNA-seq analysis:

FIG. 6A shows the significantly different expression profiles in the SP-D plus CS extract and CS only groups;

FIG. 6B shows each type of treatment that was widely separated in the principal component analysis (PCA);

FIG. 6C shows that several genes with significantly different expression levels in the SP-D plus CS group relative to the CS group involved in the regulation of oxygen species metabolic processes and inflammatory responses.

FIGS. 7A-7I show that the foamy cells were significantly increased in smokers, and COPD and ozone-exposed mice with oxLDL-treated BMDMs exhibited increased inflammation in the lung:

FIG. 7A provides the images of the foamy cells in BALF of the non-smoker and smoker, as stained with oil red O (scale bar=50 μm);

FIG. 7B shows the percentages of the foamy cells as calculated in never-smokers (non-COPD controls) (n=9), smokers included current smokers (n=4), and ex-smokers (n=9) (the data represent the mean±SEM. *p<0.05, **p<0.01, Student's unpaired t test vs. controls);

FIG. 7C shows the percentages of the foamy cells as calculated in and non-COPD controls (n=9) and smokers with COPD (n=4) (the data represent the mean±SEM. *p<0.05, **p<0.01, Student's unpaired t test vs. controls);

FIG. 7D provides the images of the ozone-induced foamy macrophages in C57BL/6J female wild-type and SP-D−/− female mice, as examined by oil red O staining (scale bar=50 μm);

FIG. 7E provides the images of the foamy cells in BMDMs pretreated with or without SP-D and then treated with LPS, LDL, and oxLDL, as examined by staining with oil red O (scale bar=50 μm);

FIG. 7F shows the C57BL/6J female mice received PBS only (PBS), medium, or LDL- or oxLDL-treated BMDMs (BM, BML, and BMO) in the air-(A-PBS, A-BM, A-BML, and A-BMO) or ozone-(O-PBS, O-BM, O-BML, and O-BMO) exposed mouse model;

FIG. 7G shows the lung function as determined using Snapshot150 perturbation on a FlexiVent (SCIREQ) in the air-(A-PBS, A-BM, A-BML, and A-BMO) or ozone-(O-PBS, O-BM, O-BML, and O-BMO) exposed mouse model (the data represent the mean±SEM. *p<0.05, **p<0.01, ***p<0.001, two-way ANOVA vs. the air group);

FIG. 7H provides the images of H&E staining of lung sections in the air-(A-PBS, A-BM, A-BML, and A-BMO) or ozone-(O-PBS, O-BM, O-BML, and O-BMO) exposed mouse model (scale bar=50 μm);

FIG. 7I shows the numbers of the total cells in BALF in the air-(A-PBS, A-BM, A-BML, and A-BMO) or ozone-(O-PBS, O-BM, O-BML, and O-BMO) exposed mouse model.

FIG. 8A-8J shows the results of naïve mice that were adoptively transferred medium-, LDL-, and oxLDL-treated BMDMs.

FIG. 8A shows that the mice were then intratracheally administered PBS or LPS on Day 2;

FIG. 8B shows that the oxLDL-treated BMDMs significantly induced cell infiltration in the lungs of LPS-treated mice but not PBS-treated mice;

FIG. 8C shows that the oxLDL-treated BMDMs significantly induced IL-1β and TNF-α production in the lungs of LPS-treated mice but not PBS-treated mice;

FIG. 8D shows that n LPS-treated mice, oxLDL-treated BMDMs resulted in higher small airway thickness than that in control mice;

FIG. 8E shows that the oxLDL-treated BMDMs induced more severe inflammation in LPS-treated mice, and the rfhSP-D-pretreated FMs reduced inflammation in the LPS treatment model;

FIG. 8F shows that the mice that received SP-D plus oxLDL-treated BMDMs had significantly reduced total cell numbers;

FIG. 8G shows the levels of TNF-α of the mice that received SP-D plus oxLDL-treated BMDMs;

FIG. 8H shows the percentages of total foamy cells;

FIG. 8I provides the images of the foamy cells;

FIG. 8J shows the alleviation of the thickened trachea in the lungs as compared to those in the oxLDL-treated BMDM groups.

FIGS. 9A-9K show that SP-D combined with oxLDL alleviated airway inflammation and improved lung function in ozone-exposed mice:

FIG. 9A provides the protocol of adoptive transfer of BMDMs in an ozone-induced C57BL/6J female mouse model of COPD; wherein the bone marrow-derived macrophages (BMDMs) were pretreated with or without SP-D (10 μg/ml) for 6 h and then treated with 50 μg/ml LDL or 50 μg/ml oxLDL for 1 day; the mice received 2× 105 live cells by intratracheal administration;

FIG. 9B shows the lung function as determined using Snapshot150 perturbation on FlexiVent (SCIREQ); wherein two-way ANOVA was performed to compare Rrs (1.46 vs. 1.91), Ers (40.00 vs. 51.27), G (10.92 vs. 6.14), and H (40.07 vs. 33.68) in the groups of mice that received oxLDL induced foamy macrophages pretreated with SP-D (O-SP-D-BMO) vs. the group of mice that received oxLDL-induced foamy macrophages without SP-D treatment (O-BMO) during 200 mg/ml methacholine challenge (the data represent the mean±SEM. *p<0.05);

FIG. 9C shows the total cell numbers in BALF in the groups of mice that received oxLDL induced foamy macrophages pretreated with SP-D (O-SP-D-BMO) vs. the group of mice that received oxLDL-induced foamy macrophages without SP-D treatment (O-BMO).

FIG. 9D provides the images of H&E staining of lung sections in BALF in the groups of mice that received oxLDL induced foamy macrophages pretreated with SP-D (O-SP-D-BMO) vs. the group of mice that received oxLDL-induced foamy macrophages without SP-D treatment (O-BMO) (scale bar=50 μm);

FIG. 9E provides the protocol of BMDM treatment during LPS plus ozone exposure;

FIG. 9F shows the lung function in BALF in the groups of mice that received oxLDL induced foamy macrophages pretreated with SP-D (O-SP-D-BMO) vs. the group of mice that received oxLDL-induced foamy macrophages without SP-D treatment (O-BMO);

FIG. 9G shows the total cell numbers in BALF in the groups of mice that received oxLDL induced foamy macrophages pretreated with SP-D (O-SP-D-BMO) vs. the group of mice that received oxLDL-induced foamy macrophages without SP-D treatment (O-BMO);

FIG. 9H provides the images of the foamy macrophages as stained with oil red O;

FIG. 9I shows the quantified results of the foamy macrophages as stained with oil red O;

FIG. 9J shows the levels of CXCL1, IL-1β, TNF-α, and IL-6 as measured by ELISA (the data represent the mean±SEM);

FIG. 9K provides the images of H&E staining of lung sections (scale bar=100 (upper) and 200 (lower) μm).

FIGS. 10A-10F show the SP-D reduced oxLDL-induced ROS production and CXCL1 expression, as well as the metabolism of several lipids, oxidative stress, and the regulation of inflammatory gene expression:

FIG. 10A provides the results of the ROS production in BMDMs pretreated with 5, 10, or 20 μg/ml SP-D, followed by treatment with 50 μg/ml LDL or oxLDL, as examined by DCFDA;

FIG. 10B provides the production of CXCL1, CCL2, and IL-6 in the BMDMs pretreated with SP-D for 1 day and then washed 3 times with PBS, and then treated with LDL or oxLDL for 1 day, wherein the supernatant was collected, as measured by ELISA;

FIG. 10C shows that the genes independently regulate the BMDMs oxidative stress; wherein the RNA expression was analyzed by RNA-seq. The left gene heatmap shows the log 2 ratios of LDL, oxLDL, SP-D+oxLDL and SP-D groups normalized to the medium control (LDL/m, oxLDL/m, SP-D+oxLDL/m, and SP-D/m); and the right gene heatmap shows the log 2 ratios of SP-D+oxLDL treatment normalized to oxLDL treatment (SP-D+oxLDL/oxLDL);

FIG. 10D shows the cytokine activity;

FIG. 10E shows the lipid metabolic processes;

FIG. 10F shows the RNA expression levels of ABCA1, ABCG1, CD36, and SR-A, as assessed by real-time quantitative PCR (qPCR) and normalized to GAPDH; wherein the results are expressed as the mean fold change compared to the medium.

FIGS. 11A-11C shows the results of the RNA-seq and PCA:

FIG. 11A shows that the gene expression profile can be clearly divided into three clusters: medium (M) and LDL treatment (LDL); oxLDL treatment (oxLDL) and SP-D plus oxLDL treatment (SPD+oxLDL) and SP-D treatment (SP-D);

FIG. 11B shows each type of treatment that was separated in the principal component analysis (PCA);

FIG. 11C shows that the oxLDL regulated the expression of genes involved in several biological processes, such as the response to oxidative stress, inflammation, and toxic substances.

FIG. 12 provides a diagram showing that the surfactant protein D inhibits lipid-laden foamy macrophages and lung inflammation in chronic obstructive pulmonary disease. SP-D deficiency exacerbates airway inflammation, lipid-laden macrophage accumulation, and emphysematous alveolar destruction in ozone- and CS-exposed lungs. Local instillation of a recombinant fragment of human surfactant protein D (rfhSP-D) alleviates oxidative stress and CSinduced airway inflammation, decreases FM formation, and causes emphysematous changes in recipient mice. The biological benefits of SP-D in ozone- and CS-induced COPD may be caused by the inhibitory effect of SP-D on ROS production in dysfunctional AMs and the restoration of lipid metabolism and cellular machinery of reverse lipid transport in lipid-laden FMs.

### DETAILED DESCRIPTION OF THE INVENTION

Unless otherwise defined herein, scientific and technical terms used herein have the meanings that are commonly understood by those of ordinary skill in the art.

As used herein, the singular forms “a”, “an”, and “the” include plural referents unless the context clearly dictates otherwise. Thus, for example, reference to “a sample” includes a plurality of such samples and equivalents thereto known to those skilled in the art.

According to the invention, increased levels of SP-D were found in the bronchoalveolar lavage fluid (BALF) and sera of ozone- and cigarette smoke (CS)-exposed mice. Furthermore, SP-D-knockout mice showed increased lipid-laden FMs and airway inflammation caused by ozone and CS exposure, as exhibited by our study cohort of chronic smokers and COPD patients. It was ascertained in the present invention that an exogenous recombinant fragment of human SP-D (rfhSP-D) prevented the formation of oxidized low-density lipoprotein (oxLDL)-induced FMs in vitro and reversed the airway inflammation and emphysematous changes caused by oxidative stress and CS exposure in vivo. SP-D upregulated bone marrow-derived macrophage (BMDM) expression of genes involved in countering the oxidative stress and lipid metabolism perturbations induced by CS and oxLDL, demonstrating the crucial roles of SP-D in the lipid homeostasis of dysfunctional alveolar macrophages caused by ozone and CS exposure in experimental mouse emphysema. Accordingly, the present invention provides a therapy of SP-D in patients with COPD.

As used herein, the term, “surfactant protein-D” or “SP-D” refers to a hydrophilic lung surfactant protein secreted by alveolar type II cells and recycled by alveolar macrophages (AMs) [5]. SP-D plays a significant role in the protection against COPD by reducing the production of oxidants [6] and inflammatory responses in AMs [7] and increasing apoptotic cell clearance [8].

In the invention, a native SP-D, a fragment or a recombinant fragment of human SP-D (rfhSP-D) can be used.

One example of the native SP-D is a wild type human SP-D, consisting of the amino acid sequence below (SEQ ID NO: 1):

MLLFLLSALVLLTQPLGYLEAEMKTYSHRIMPSACTLVMCSSVESGLPGRD
GRDGREGPRGEKGDPGLPGAAGQAGMPGQAGPVGPKGDNGSVGEPGPKGD
TGPSGPPGPPGVPGPAGREGPLGKQGNIGPQGKPGPKGEAGPKGEVGAPGM
QGSAGARGLAGPKGERGVPGERGVPGNTGAAGSAGAMGPQGSPGARGPPG
LKGDKGIPGDKGAKGESGLPDVASLRQQVEALQGQVQHLQAAFSQYKKVEL
FPNGQSVGEKIFKTAGFVKPFTEAQLLCTQAGGQLASPRSAAENAALQQLV
VAKNEAAFLSMTDSKTEGKFTYPTGESLVYSNWAPGEPNDDGGSEDCVEIF
TNGKWNDRACGEKRLVVCEF.

One example of rfhSP-D is the fragment consisting of the amino acid sequence the amino acid sequence below (SEQ ID NO: 2), which is a fragment of the 199th-375th residues of the native SP-D, having 177 amino acid, and a modification at the 200th residue to Serine (Ser, S) from Proline (Pro, P):

GSPGLKGDKGIPGDKGAKGESGLPDVASLRQQVEALQGQVQHLQAAFSQY
KKVELFPNGQSVGEKIFKTAGFVKPFTEAQLLCTQAGGQLASPRSAAENA
ALQQLVVAKNEAAFLSMTDSKTEGKFTYPTGESLVYSNWAPGEPNDDGGS
EDCVEIFTNGKWNDRACGEKRLVVCEF.

The term “subject” as used herein refers to a human and/or a non-human animal, such as companion animals (e.g., dogs, cats, etc.), farm animals (e.g. cattle, sheep, pigs, horses, etc.), or experimental animals (e.g., rats, mice, guinea pigs, etc.).

The term “treat,” “treating” or “treatment” as used herein refers to the application or administration of a composition including one or more active agents to a subject afflicted with a disease, a symptom or conditions of the disease, or a progression of the disease, with the purpose to cure, heal, alleviate, relieve, alter, remedy, ameliorate, improve, or affect the disease, the symptoms or conditions of the disease, the disabilities induced by the disease, or the progression of the disease.

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function.

The term “pharmaceutically acceptable carrier” used herein refers to a carrier(s), diluent(s) or excipient(s) that is acceptable, in the sense of being compatible with the other ingredients of the formulation and not deleterious to the subject to be administered with the pharmaceutical composition. Any carrier, diluent or excipient commonly known or used in the field may be used in the invention, depending to the requirements of the pharmaceutical formulation. Said carrier may be a diluent, vehicle, excipient, or matrix to the active ingredient.

The present invention is further illustrated by the following examples, which are provided for the purpose of demonstration rather than limitation.

### Example

### Materials and Methods

### Mice

Eight-week-old female BALB/cByJNarl, C57BL/6, and SP-D mice were purchased from the National Laboratory Animal Center (Taiwan), consisting of the amino acid sequence of SEQ ID NO:1. The mice were housed in the Laboratory Animal Center of the College of Medicine National Cheng Kung University. Sftpd-knockout (SP-D−/−) mice were generated by CRISPR/Cas 9 at the National Laboratory Animal Center (Tainan, Taiwan). The animal studies were approved by the University Institutional Animal Care and Use Committee (IACUC No. 106243, 109006, and 109266), National Cheng Kung University (NCKU). In the ozone-induced model, the mice were exposed to ozone (3 and 10 ppm) or air for 3 h per day, 3 times per week for 3 weeks. SP-D treatment involved intratracheal administration of 20 μg of native SP-D once per week for 3 weeks. In the CS-induced mouse model, the mice were exposed to 6 cigarettes for 1 h, 5 times per week for 6 weeks. The animals were then sacrificed by intraperitoneal injection with 90 mg/kg b.w. pentobarbiturate and exsanguination. The mechanical properties of the lung were determined by using the Scireq Flexivent apparatus (SCIREQ, Montreal, QC, Canada). In the BMDM adoptive transfer experiment, the mice were intratracheally administered PBS (vehicle control), 4×105 BMDMs (3 weeks of ozone exposure) or 5×105 BMDMs (LPS and ozone exposure). These mice were then allowed 1 day of rest. On Day 2, the mice were treated with ozone or LPS as described above. After being sacrificed as described above, the mechanical properties of the lung were determined using the Scireq Flexivent apparatus (flexiVent; SCIREQ, Montreal, QC, Canada). The lung tissue was fixed in paraformaldehyde (Sigma-Aldrich Co., USA) for histology.

Collection of Cells from BALF

BALF was collected by 2 instillations of 1 ml of cold saline into the trachea. The BALF was centrifuged at 300×g for 5 min at 4° C. The cells were incubated with RBC lysis buffer (eBioscience). After 3 min, PBS was added, and the samples were centrifuged at 300×g for 5 min at 4° C. Total cell counts were determined by a hemocytometer. Next, the cells were prepped with a cytospin and then Liu-stained (Tonyar Biotech, Tao Yuan, Taiwan). Immune cell types were identified by microscopy and the enumeration of 200 cells. Cells stained with oil red O (Sigma-Aldrich Co., USA) were counted to determine red-stained foam cells.

### ELISA

Cytokines (TNF-α, IL-6, CXCL1, CCL2, and IL-1β) in BALF or cell supernatants were measured by ELISA (R&D System, Minneapolis, MN, USA). SP-D levels in BALF and serum was measured by a monoclonal antibody based on the Mouse SP-D DuoSet ELISA kit (Catalog No. DY6839-05, R&D System, Minneapolis, MN, USA).

### Lung Histology and Immunohistochemistry

H&E staining was performed according to the manufacturer's protocols (Sigma-Aldrich Co., USA). Paraffin-embedded sections were heated to 65° C. for 20 min and deparaffinized in Histoclear (3×10 min). The sections were rehydrated in 100%, 95%, 90%, and 70% alcohol and water for 10 min. Next, the slides were placed in retrieval solution (Dako, Carpinteria, CA) and heated in a microwave for 15 min. After being cooled to room temperature for 30 min, immunohistochemistry was performed using an IHC kit (Dako, Carpinteria, CA) and Nrf2 antibodies (Abcam, Cambridge, United Kingdom). Images were acquired using an Olympus BX50 light microscope (Olympus, Center Valley, PA, USA) equipped with an Optronics MagnaFire digital camera (Optronics, Inc., Muskogee, OK, USA). Images of lung sections were captured and assessed for the mean linear intercept (MLI), which was calculated by dividing the total length of the lung section by the total number of intercepts with alveolar septal walls (scale bar=100 μm) [38].

### Imaging ROS

The mice received L-012 by intraperitoneal administration. After 10 min, the mice were euthanized, and their lung tissue was examined by an IVIS Lumina LT Series III (PerkinElmer, USA).

### Purification of Human SP-D and Expression of a Recombinant Fragment of Human SP-D

Native SP-D was purified from pooled amniotic fluid, which was filtered through a 0.45-nm membrane. The filtrate was incubated with maltose Sepharose at 4° C., and SP-D protein was purified using a packed maltose Sepharose column by ÄKTA Start (GE Healthcare Life Sciences, USA) [39]. Recombinant human SP-D was produced and purified as described elsewhere [40] with minor modifications. To assess the purity, purified rfhSP-D was separated by SDS-PAGE. LPS was removed using Acrodisc units with a Mustang E Membrane (PALL Corporation, NY, USA). BMDMs were treated with recombinant full-length SP-D (R&D System, Minneapolis, MN, USA). The rfhSP-D has the amino acid sequence of SEQ ID NO:2.

### Collection of Bronchoalveolar Lavage Fluid

Bronchoalveolar lavage (BAL) samples were collected from the participants at the National Cheng Kung University Hospital (IRB approval IRB: B-ER-109-016). The procedures were performed according to the recommended practice guideline procedures. A total of 9 never-smoker controls, 4 current smokers and 9 ex-smokers were recruited for the study. One current smoker and 3 ex-smokers were diagnosed with COPD by using the GOLD criteria with clinical correlations or by CT scans. After wedging in the targeted segmental bronchus, BAL of the unaffected lung was performed using a fiberoptic bronchoscope. The first was an installed aliquot that was disposed to avoid contamination of the bronchial sections. The flowing aliquots were instilled and withdrawn sequentially. Next, 1 ml of BAL was centrifuged at 300×g for 5 min at 4° C. The cells were incubated with RBC lysis buffer (eBioscience). After 3 min, PBS was added, and the samples were centrifuged at 300×g for 5 min at 4° C. The BAL cells were prepared on a microscope slide and further stained with Oil red O.

### Cell Treatment

A549 or BES-2B cells were pretreated with 1, 2, 5, and 10 μg/ml SP-D for 5 h, followed by 25 μM H₂O₂ for 2 h.

### Western Blotting

Samples were loaded on a 10% Tris-glycine polyacrylamide gel. After electrophoretic separation, the proteins were transferred to a polyvinylidene difluoride (PVDF) membrane. The blots were blocked in 5% dry milk blocking buffer and then washed. The following antibodies were used: Nrf2 (1:1000, GeneTex, USA), β-actin (1:5000, GeneTex, USA), and an antirabbit secondary antibody (1:10,000, GeneTex, USA). The probed blots were stained with an ECL western blotting substrate kit (PerkinElmer, USA) and detected by a UVP BioSpectrum Imaging System.

### Flow Cytometry

Approximately 106 cells were collected. Apoptotic cells were immediately assessed with a FITC Annexin V Apoptosis Detection Kit (BD Biosciences, San Jose, CA, USA) and a BD FACScan (BD Biosciences, San Jose, CA, USA).

Preparation of Oxidized LDL (oxLDL)

Human LDL (Prospec) was oxidized by CuSO4 (Sigma-Aldrich Co., USA) at 37° C. for 1 day. The oxidation was stopped by adding EDTA (Sigma-Aldrich Co., USA). The oxLDL was dialyzed in PBS for 2 h and exchanged three times. The oxLDL was filtered through a 0.22-μm pore hydrophilic PVDF membrane (Merck Millipore, USA). The concentration of oxLDL was measured by using a Bio-Rad protein assay kit (Bio-Rad, Hercules, CA, USA).

### Preparation of BMDMs

Bone marrow cells were obtained from the thigh bone and cultured in 10 ng/ml M-CSF for 1 week. The medium was renewed every 3 days. After 1 week, these cells transformed into macrophages, as described elsewhere [41]. The cells were distributed at 1×106 per well and treated with 50 μg/ml LDL or 50 μg/ml oxLDL. The cells were pretreated with or without SP-D (10 μg/ml) for 1 day and washed with PBS before being treated with oxLDL. After 1 day, the cells were collected and washed with PBS.

### RNA-Seq Analysis

BMDMs were pretreated with or without SP-D (10 μg/ml) for 6 h and then washed with PBS. The cells were treated with 0.3% cigarette extract for 6 h and then collected. In the cigarette extract experiment, BMDMs were pretreated with or without SP-D (10 μg/ml) for 6 h and then washed with PBS. The cells were treated with 0.3% CS for 6 h. In the oxLDL analysis, the BMDMs were pretreated with or without SP-D (10 μg/mL) for 1 day and then washed with PBS. These cells were treated with oxLDL (50 μg/mL) for 1 day and then collected. Total RNA was extracted using TRIzol reagent (Invitrogen, USA) according to the manufacturer's instructions. Purified RNA was quantified at 260 nm using an ND-1000 spectrophotometer (Nanodrop Technology, USA), and the quality was assessed using a Bioanalyzer 2100 (Agilent Technology, USA) with an RNA 6000 LabChip kit (Agilent Technology, USA). All RNA procedures were carried out according to Illumina protocols. The Agilent SureSelect Strand-Specific RNA Library Preparation Kit was used for library construction followed by AMPure XP bead (Beckman Coulter, USA) size selection. The sequence was determined using sequencing-by-synthesis technology (Illumina, USA). The sequencing data (FASTQ reads) were generated using Welgene Biotech's pipeline based on Illumina's base-calling program bcl2fastq v2.20. The mRNA profiling-seq data generated from the treated groups were normalized to the data from the medium control. After a pairwise comparison, genes with ≥2-fold up- and downregulation in each comparison were selected for analysis. Differential expression analysis was performed using cuffdiff (cufflinks v2.2.1) with genome bias detection/correction and Welgene in-house programs. The differentially expressed genes of each experiment were subjected to the enrichment test for functional assays using clusterProfiler v3.6. Genes associated with oxidative stress, inflammation, and lipid metabolism pathways were identified by analytical filters and used to further draw heatmaps using GraphPad Prism (GraphPad Software).

### Real-Time PCR Analysis

Total RNA was isolated using TRIzol RNA Isolation Reagents (Thermo Fisher Scientific), and 1 μg was treated with DNase I (Promega, USA) and reversetranscribed (Promega, USA). qPCRBIO SyGreen Mix (PCR Biosystems) (PCR Biosystems, London, UK) was used to amplify ABCA-1, ABCG-1, CD36, and SR-A by qPCR using the following primers:

```
(1) ABCA-1
  (SEQ ID NO: 3)
fwd: 5′-GCAGATCAAGCATCCCAACT-3′,
  (SEQ ID NO: 4)
rev: 5′-CCAGAGAATGTTTCATTGTCCA-3′;
(2) ABCG-1
  (SEQ ID NO: 5)
fwd: 5′-GGGTCTGAACTGCCCTACCT-3′,
  (SEQ ID NO: 6)
rev: 5′-TACTCCCCTGATGCCACTTC-3′;
(3) CD36
(SEQ ID NO: 7)
fwd: 5′-TTGTACCTATACTGTGGCTAAATGAGA-3,
  (SEQ ID NO: 8)
rev: 5′-TCTACCATGCCAAGGAGCTT-3′;
(4) SR-A
  (SEQ ID NO: 9)
fwd: 5′-GCATCCCTTCCTCACAGC-3′,
  (SEQ ID NO: 10)
rev: 5′-AATGAGGGCAGCCTTGAA-3′;
and
(5) GAPDH
  (SEQ ID NO: 11)
fwd: 5′-GGTCATCCATGACAACTTTGG C-3′,
(SEQ ID NO: 12)
rev: 5′-TGGATGCAGGGATGATGTTCTG-3′
```

Cellular ROS Assay with DCFDA

A cellular ROS assay was conducted using the DCFDA/H2DCFDA-Cellular ROS Assay Kit (Abcam, Cambridge, United Kingdom). The cells were seeded overnight on a 96-well culture plate (2.5×104 cells/well) with a clear flat bottom and black sides. The cells were washed with 1× buffer and then stained with diluted DCFDA solution for 45 min at 37° C. The cells were then treated with different doses of H₂O₂ and 50 μM tert-butyl hydrogen peroxide (TBHP, as a positive control) in 1× buffer supplemented with 10% FBS for 4 h using a measured plate in Ex/Em=485/535 nm endpoint mode using FlexStation 3 (Molecular Devices, LLC.).

### Statistical Analysis

Cell numbers, ELISA data, and mRNA expression were analyzed by Student's unpaired t test. The response to methacholine challenge under various conditions was compared by two-way ANOVA. The mean±SEM are presented as a bar chart using GraphPad Prism 8. A value of p<0.05 indicates statistical significance.

### Results

Increased SP-D Expression in Mice with Ozone-Induced COPD

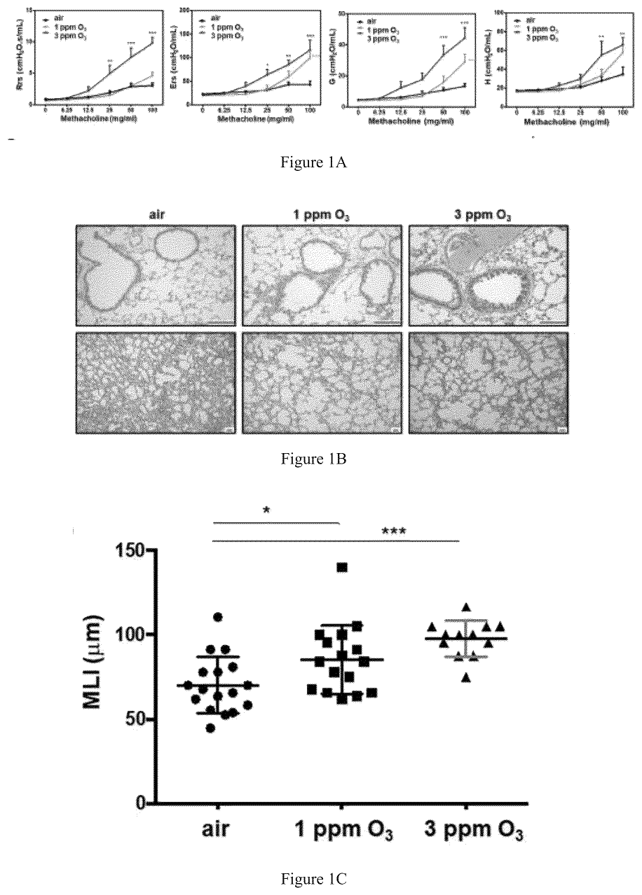

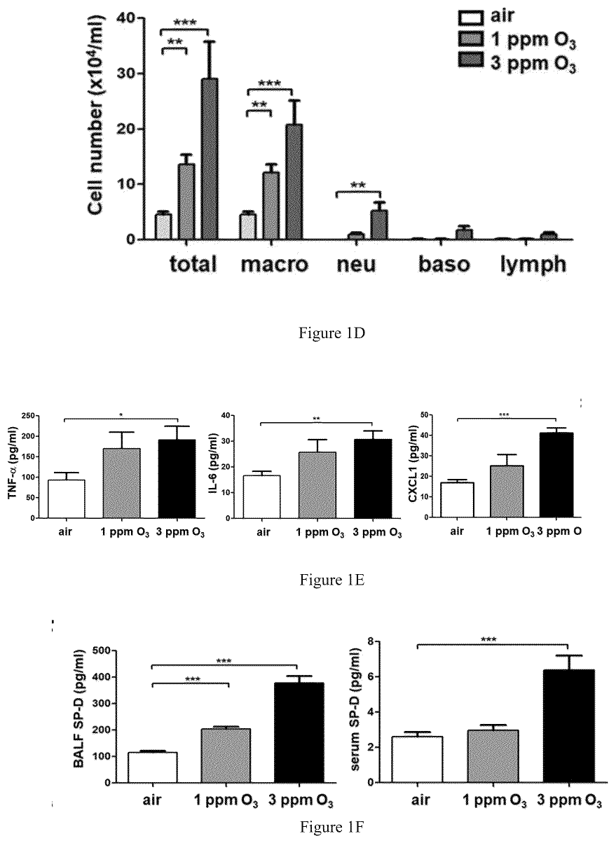

We evaluated SP-D levels in a mouse model of COPD. The mice were subjected to chronic ozone exposure that directly destroyed alveolar structure and induced airway inflammation, resulting in chronic lung disease. After 3 weeks of ozone or air exposure, the mice were administered increasing doses of methacholine. In ozone-exposed mice, 1 and 3 ppm ozone increased respiratory system elastance (Ers) and lung tissue damping (G), while 3 ppm ozone also significantly increased respiratory-flow resistance (Rrs) and lung-tissue elastance (H) compared to those in air-exposed mice (FIG. 1A). Lung histology showed severe bronchial inflammation and alveolar space enlargement in ozone-exposed mice but not in control mice (FIG. 1B). Ozone increased the mean linear intercept (MLI) in a dose-dependent manner (FIG. 1C). Ozone exposure not only increased total inflammatory cell infiltration in the lung, particularly that of macrophages and neutrophils (FIG. 1D), but also promoted the secretion of TNF-α, IL-6, and CXCL1 in bronchoalveolar lavage fluid (BALF) (FIG. 1E). These results indicate that chronic exposure to 3 ppm ozone for 3 weeks successfully induced airway inflammation and alveolar emphysema in mice, and the levels of SP-D in the serum and BALF were significantly increased in ozone-exposed mice (FIG. 1F).

Increased SP-D Expression in Mice with CS-Induced COPD

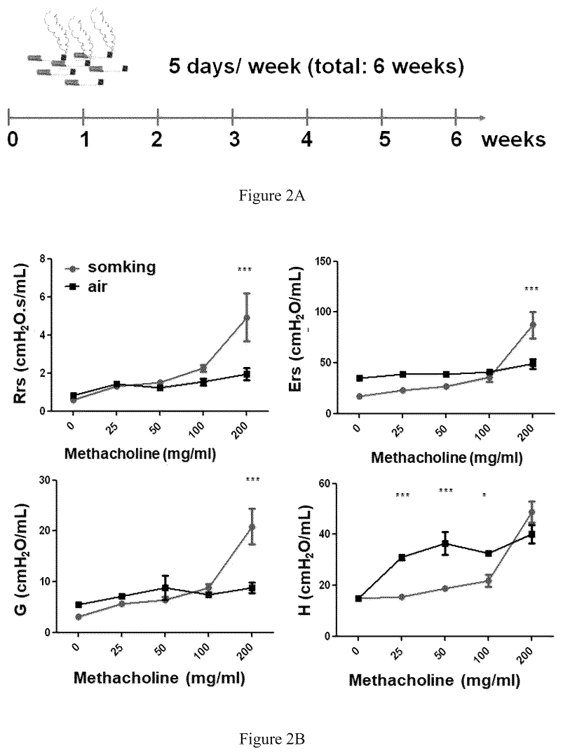

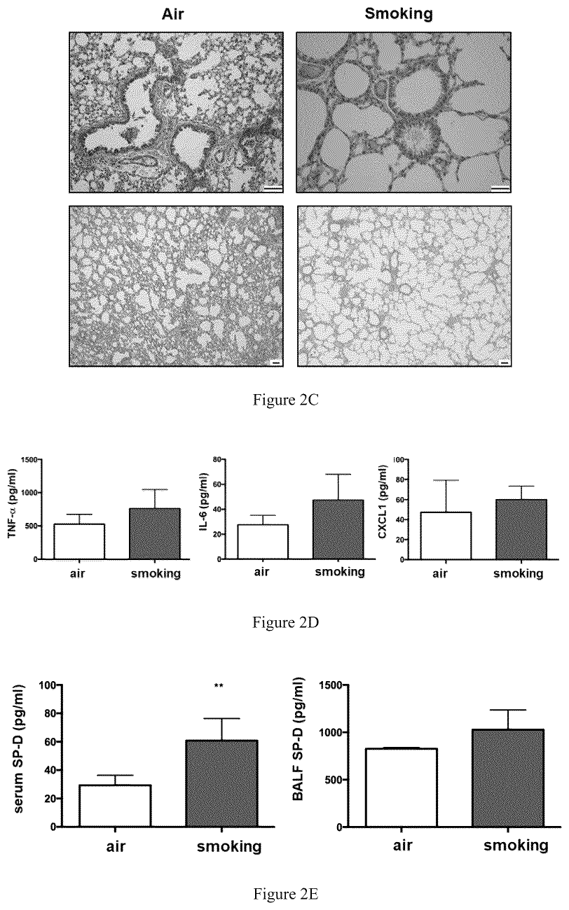

The SP-D levels were evaluated in a mouse model of CS-induced COPD, as comparable to the etiologic inducer of COPD in human. The mice were exposed to the smoke of 6 burning cigarettes for 1 h, 5 times per week for 6 weeks (FIG. 2A). CS exposure significantly increased the Rrs, Ers, and G after 200 mg/mL methacholine challenge compared to those in control mice exposed to air (FIG. 2B). Lung histological sections (H&E staining) showed that CS exposure increased mucus production in the bronchioles and caused emphysema (FIG. 2C). The expression of cytokines and chemokines (TNF-α, IL-6, and CXCL1) increased but did not achieve statistical significance after CS exposure (FIG. 2D). CS exposure increased the level of SP-D in the serum but did not cause any significant change in the BALF (FIG. 2E). These results indicated that 6 weeks of CS exposure induced emphysema and increased mucus production and inflammatory cytokine expression in the lungs.

### Enhanced Airway Inflammation and Emphysematous Changes in Ozone-Induced SP-D-Knockout Mice

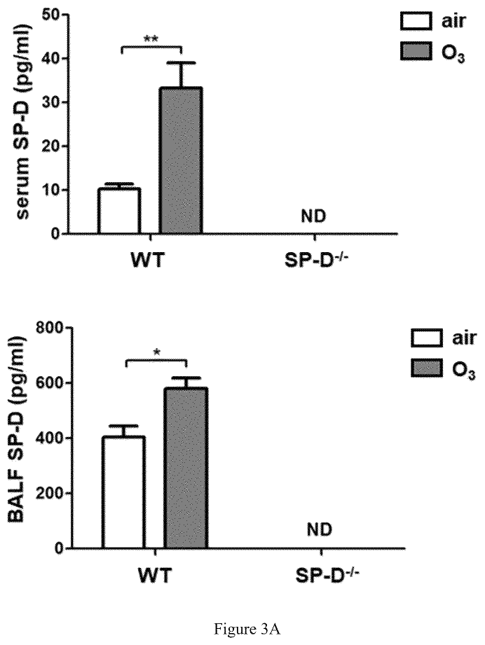

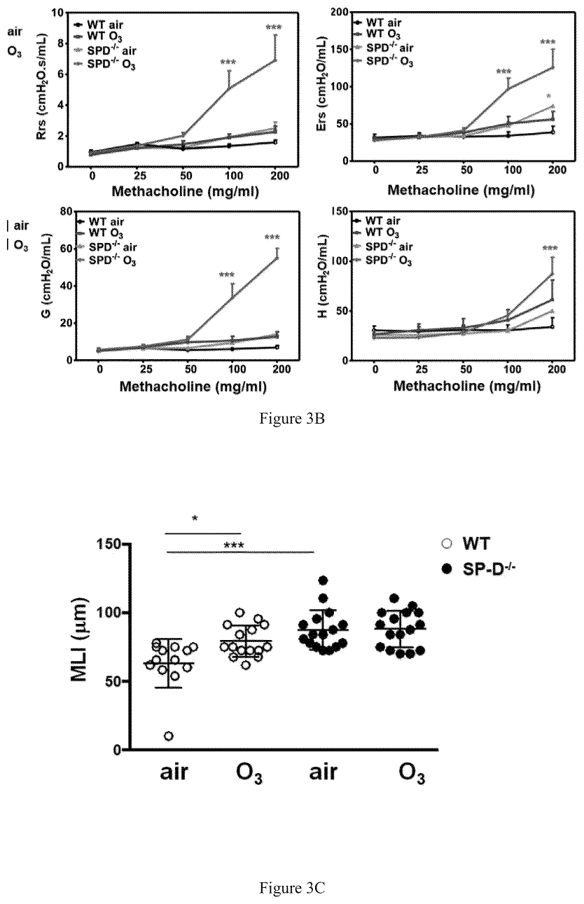

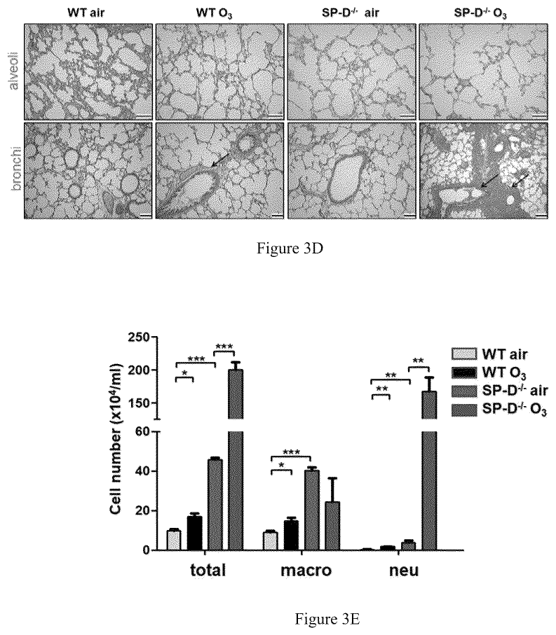

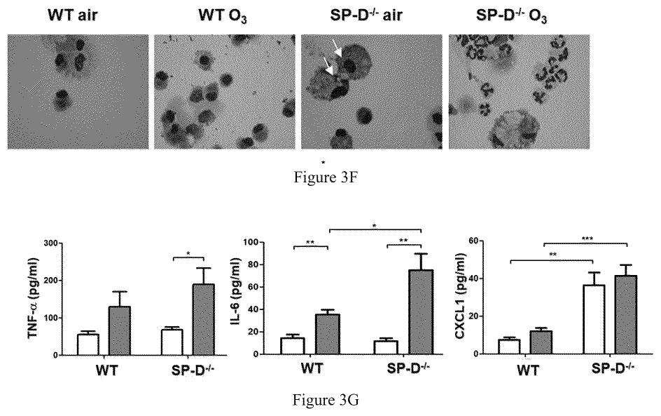

To investigate the functional role of SP-D in COPD, we used CRISPR was used to generate sftpd-knockout (SP-D−/−) mice and confirmed the absence of SP-D in both the serum and BALF (FIG. 3A). All lung function parameters (Rrs, Ers, G, and H) were significantly increased in ozone-exposed SP-D−/− mice compared to wild-type mice (FIG. 3B). Consistent with previous reports, air-exposed SP-D−/− mice exhibited larger alveolar spaces in the lung, as indicated by the increased MLI compared to that of wild-type mice (FIG. 3C). Ozone exposure further enhanced the development of emphysema and the infiltration of inflammatory cells, particularly neutrophils, in the lungs of SP-D−/− mice (FIG. 3D). The total number of cells and neutrophils significantly increased in the BALF of SP-D−/− mice exposed to ozone compared to that of air-treated SP-D-deficient mice and wild-type mice (FIG. 3E). Moreover, the BALF of SP-D−/− mice had a higher number of stained lipid-laden FMs (FIG. 3F). Airexposed SP-D−/− mice exhibited significantly increased CXCL1 levels in BALF compared to air-exposed wild-type mice. In addition, ozone exposure significantly increased the levels of IL-6 and CXCL1 in the BALF of SP-D−/− mice compared to wild-type mice (FIG. 3G), which suggested that SP-D depletion promoted airway inflammation and emphysematous changes in the lungs of ozone-exposed mice.

### Exogenous SP-D Improved Lung Function and Attenuated Airway Inflammation in Ozone-Exposed Mice

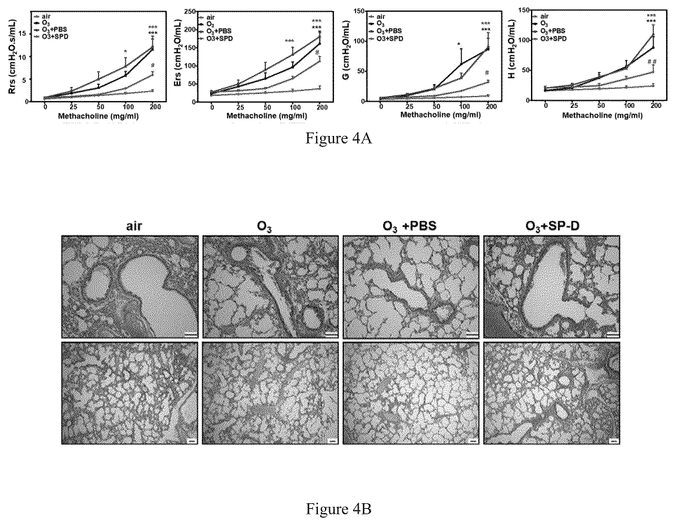

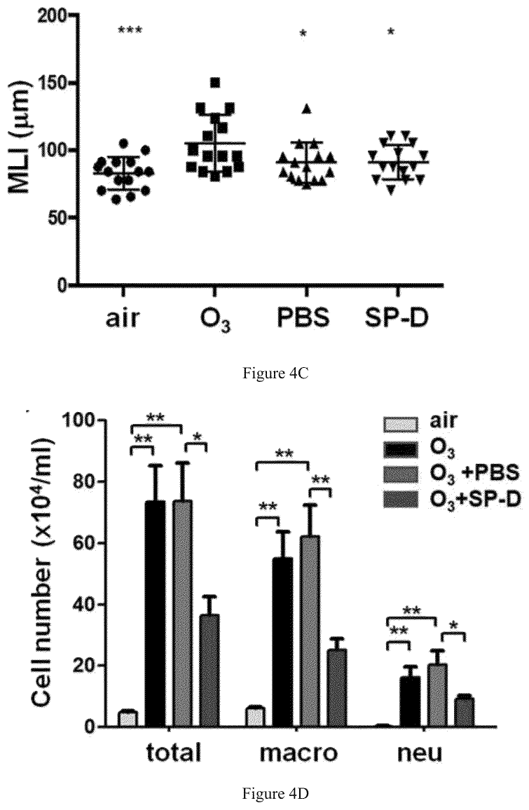

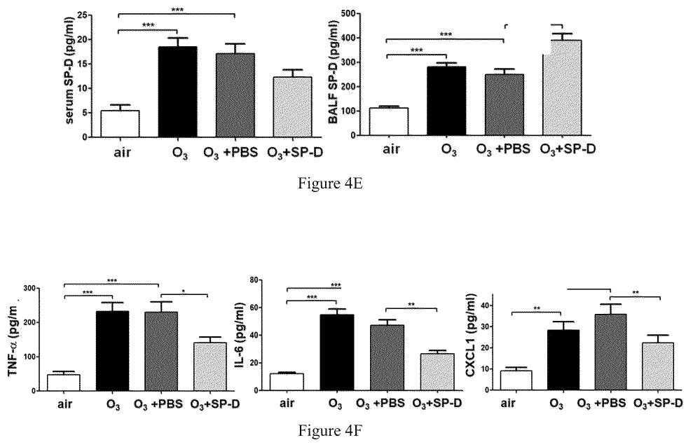

Native SP-D (25 μg) purified from amniotic fluid was intratracheally administered to ozone-exposed mice once per week for 3 weeks. Exogenous SP-D significantly improved lung function, as demonstrated by reductions in Rrs, Ers, G, and H, compared with those in untreated mice (FIG. 4A). SP-D alleviated inflammation in the alveoli and airways of ozone-exposed mice (FIG. 4B), reduced lung emphysema (as shown by the lower MLI (FIG. 4C)), and decreased inflammatory cell infiltration, particularly that of macrophages and neutrophils, in the BALF of SP-D-treated ozone-exposed mice (FIG. 4D). In addition, exogenous administration of native human SPD significantly increased the levels of SP-D in the BALF but not the sera of treated mice (FIG. 4E). TNF-α, IL-6, and CXCL1 levels were significantly decreased in BALF after SP-D treatment (FIG. 4F). These data support the hypothesis that SP-D protects against airway inflammation and emphysematous changes associated with COPD.

### SP-D Decreased ROS Production by Upregulating Nrf2 and Prevented ROS-Induced Early Apoptosis of Epithelial Cells

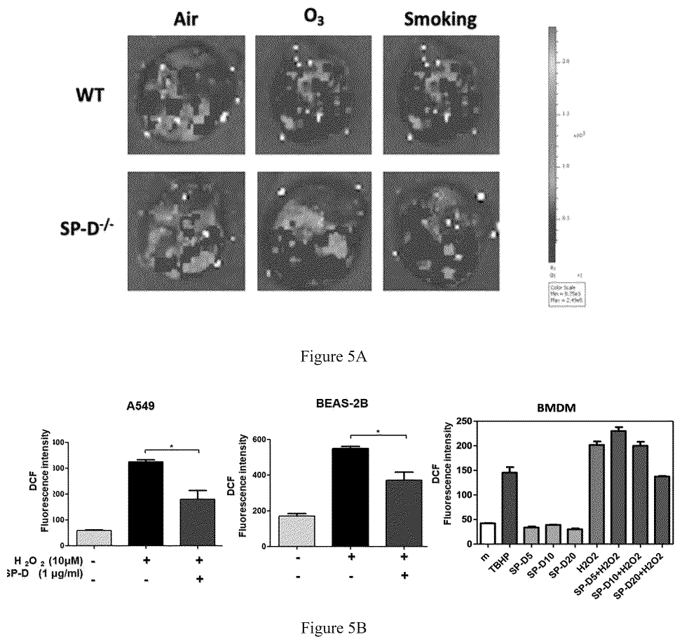

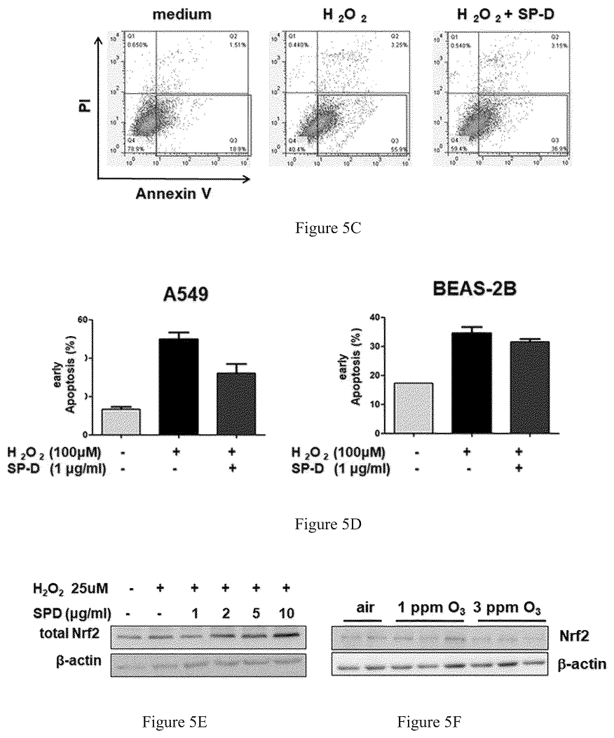

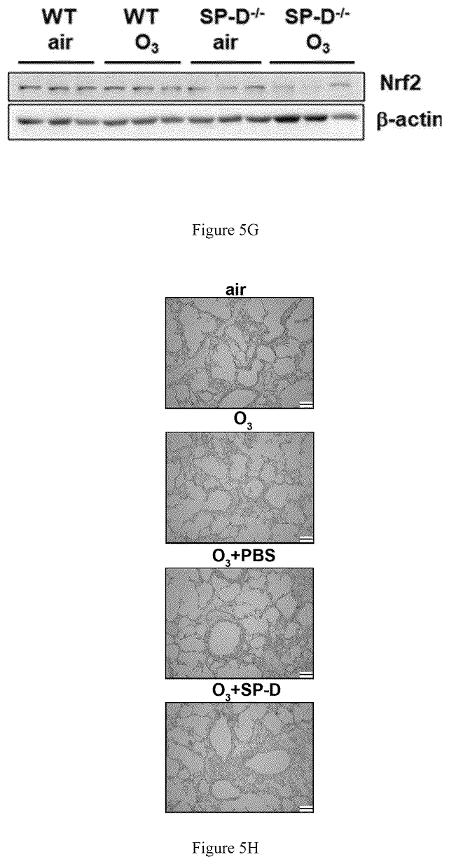

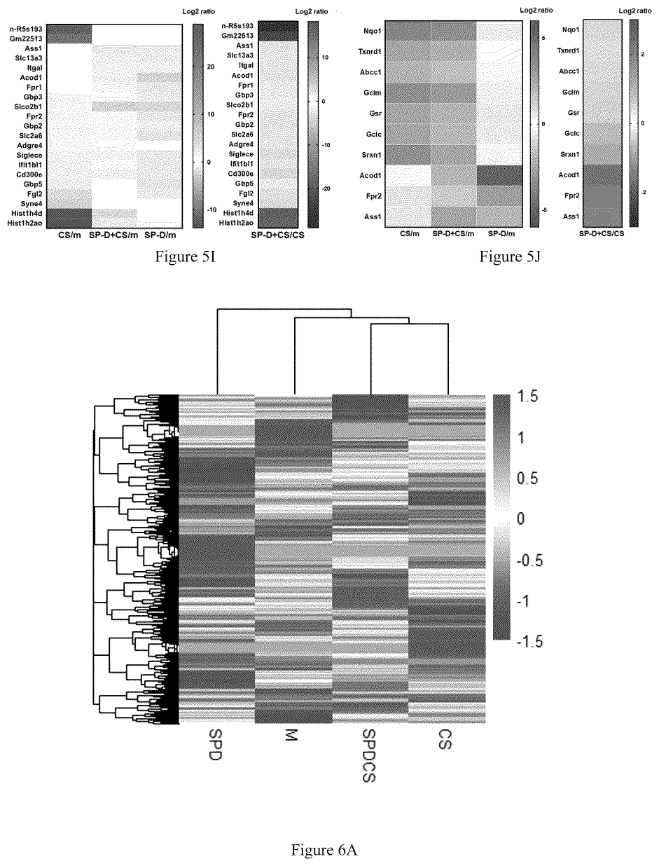

As shown in FIG. 5, SP-D deficiency increased the level of ROS in the lungs of ozone- and CS-exposed SP-D-knockout or wild-type mice. Ex vivo bioluminescence imaging showed that SP-D−/− mice CS exposed conditions compared to wild-type mice, indicating that SP-D was essential for preventing ROS production in the lung (FIG. 5A). In vitro studies on cell lines also showed that pretreatment with SP-D reduced ROS production in A549 and BEAS-2B cells stimulated with 10 μM H₂O₂ and dose-dependently reduced ROS levels in SP-D-pretreated, H2O2-stimulated bone marrow-derived macrophages (BMDMs) (FIG. 5B). To examine whether SP-D reduced hydrogen peroxide-induced programmed cell death, A549 cells were pretreated with 1 μg/ml SP-D for 5 h followed by 10 μM H2O2, and early-stage apoptosis was assessed (annexin V+ and PI−) by flow cytometry. H2O2 increased the annexin V+ and PI− populations of A549 and BEAS-2B cells, while SP-D pretreatment significantly decreased the annexin V+ and PI populations of A549 cells but not BEAS-2B cells (FIG. 5C, and FIG. 5D).

Nuclear factor erythroid 2-related Factor 2 (Nrf2) is a basic leucine zipper (bZIP) protein that can regulate the expression of antioxidant proteins that protect against oxidative damage triggered by injury and inflammation [20]. To examine whether SP-D decreased ROS production through Nrf2, A549 cells were pretreated with various doses of SP-D and then stimulated with H₂O₂. We found that pretreatment with 5 and 10 g/mL SP-D increased the expression of Nrf2 (FIG. 5E). In ozone-exposed (3 ppm) wild-type mice, Nrf2 protein expression in the lung was lower than that in air-exposed mice (FIG. 5F). Air-exposed SP-D−/− mice exhibited lower Nrf2 protein significantly reduced Nrf2 expression in SP-D−/− mice (FIG. 5G). The histochemistry results showed that exogenous administration of SP-D increased the expression of Nrf2 in the lungs of ozone-exposed mice compared to untreated mice (FIG. 5H).

### SP-D Upregulated Oxidative and Lipid Metabolism Gene Expression in CS Extract-Treated BMDMs

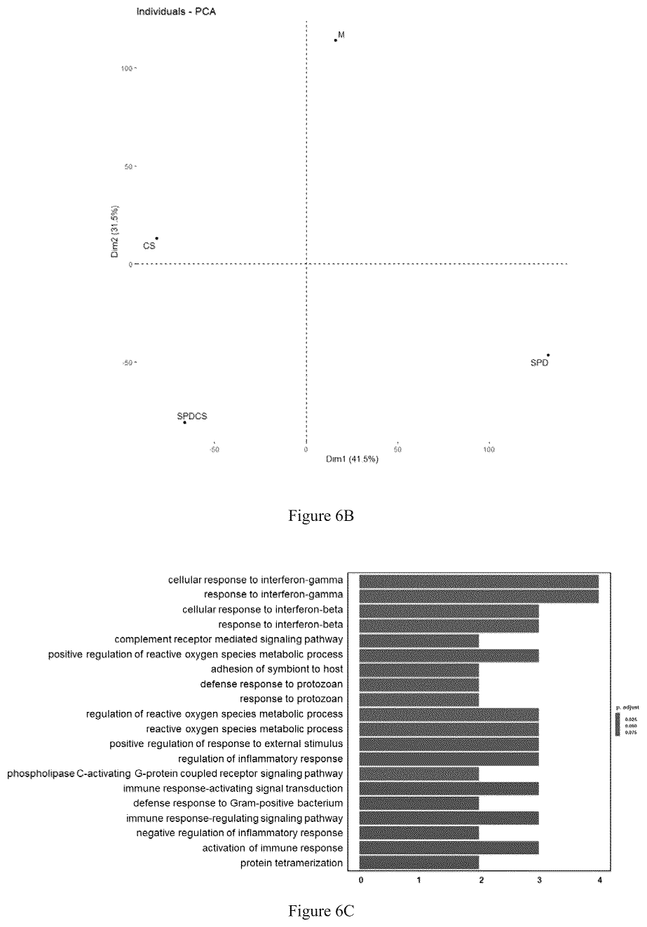

To clarify the oxidative stress-related genes that are regulated by SPD, we performed RNA-seq analysis on BMDMs treated with the medium, SP-D, CS extract, or SP-D plus CS extract. The heatmap showed significantly different expression profiles in the SP-D plus CS extract and CS only groups (FIG. 6A). Each type of treatment was widely separated in the principal component analysis (PCA) (FIG. 6B). SP-D plus CS extract changed 21 genes compared with CS alone (FIG. 5I). Several genes with significantly different expression levels in the SP-D plus CS group relative to the CS group were involved in the regulation of oxygen species metabolic processes and inflammatory responses (FIG. 6C). CS extract-treated BMDMs had 8 increased and 2 decreased genes associated with ROS metabolic processes compared with the medium control (FIG. 5J left). However, SP-D pretreatment reversed 3 of the effects of CS inhibition, including those on formyl peptide receptor 2 (Fpr2), aconitate decarboxylase 1 (Acod1, also called Irg-1), and arginosuccinate synthetase 1 (Ass1) (FIG. 5J right).

Increased FMs in Patients with COPD and SP-D Deficient Mice

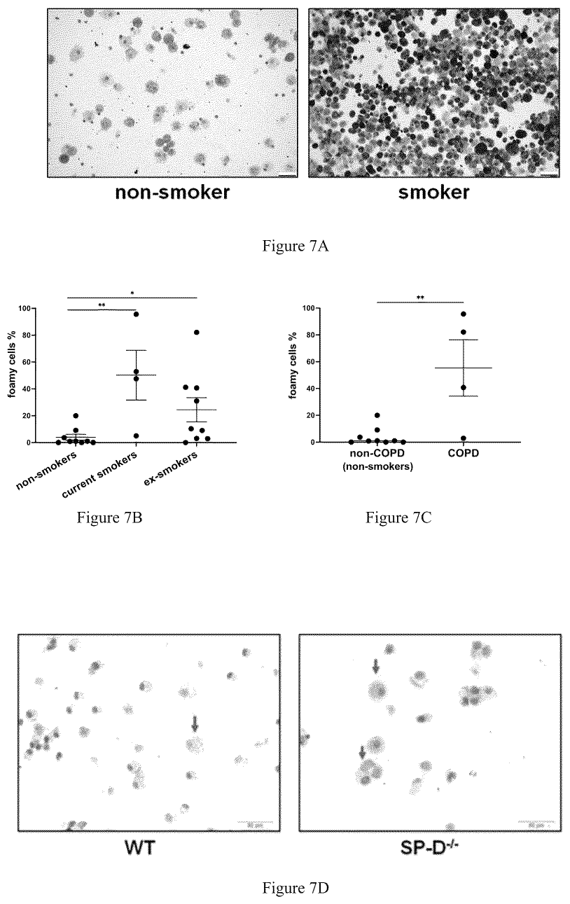

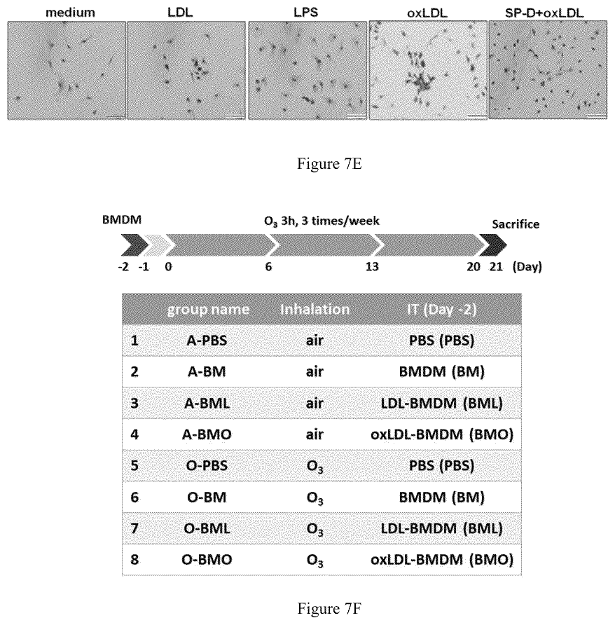

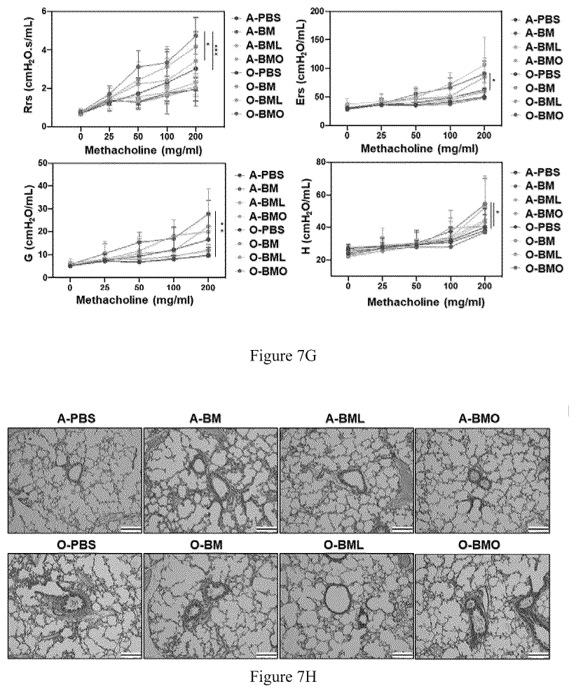

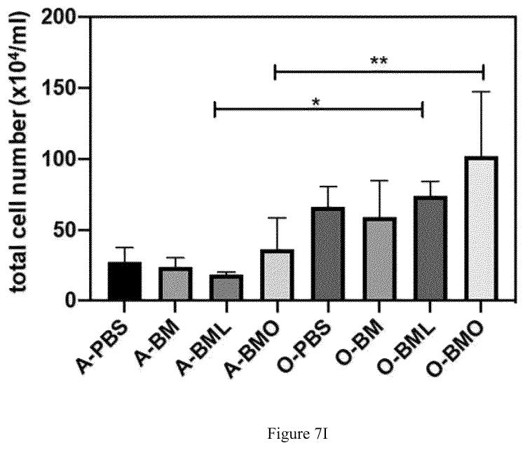

Our COPD patient cohort demonstrated that not only COPD patients but also smokers (both current and ex-smokers) had significantly increased oil-red-O-stained, lipid-laden FMs in BALF compared to nonsmokers (FIGS. 7A-7C). In ozone-induced COPD, SP-D−/− mice exhibited more FMs than wild-type mice (FIG. 7D). In vitro, oxLDL-treated BMDMs exhibited more FMs than nonoxidized LDL- or LPS-treated BMDMs. Moreover, SP-D pretreatment of BMDMs decreased the number of oxLDL-induced FMs (FIG. 7E).

### FM Induced Airway Inflammation in Naïve Mice

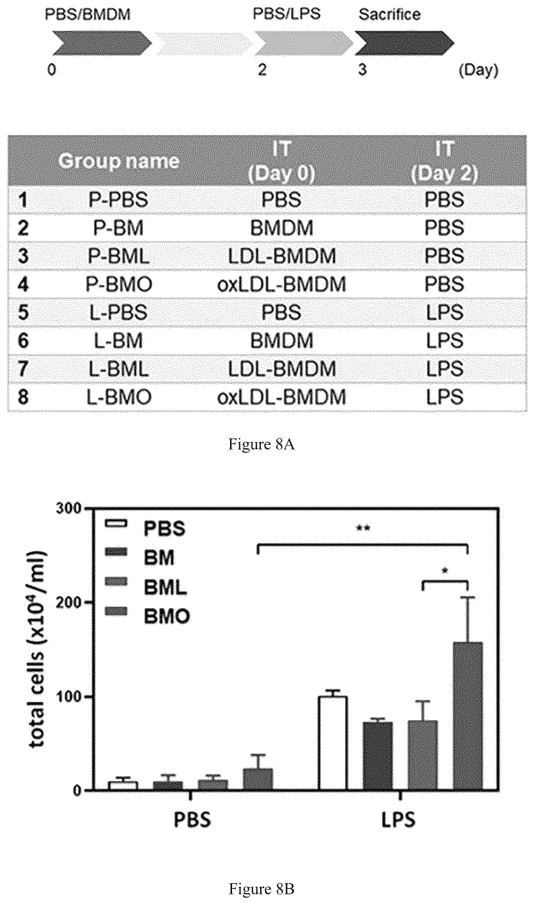

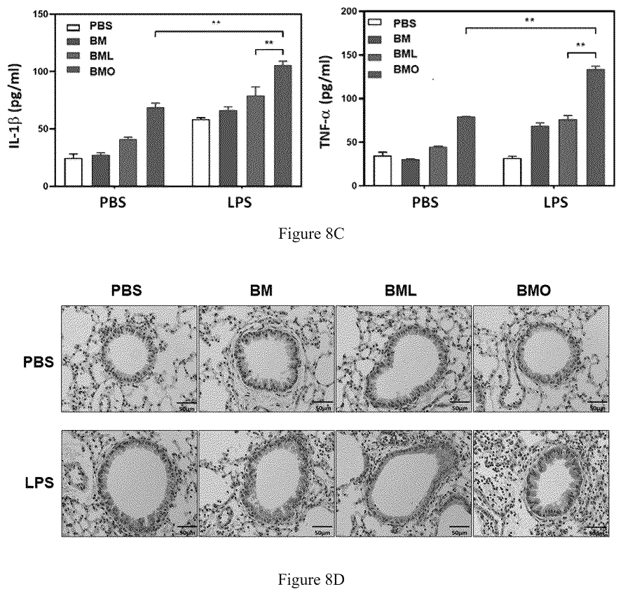

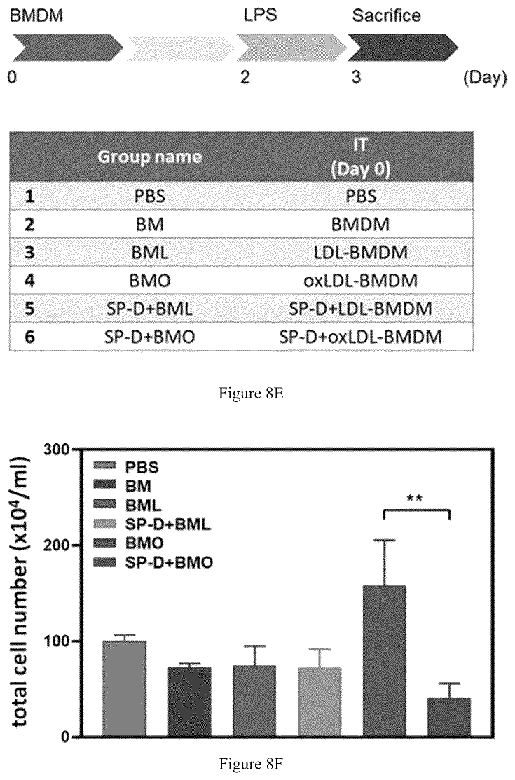

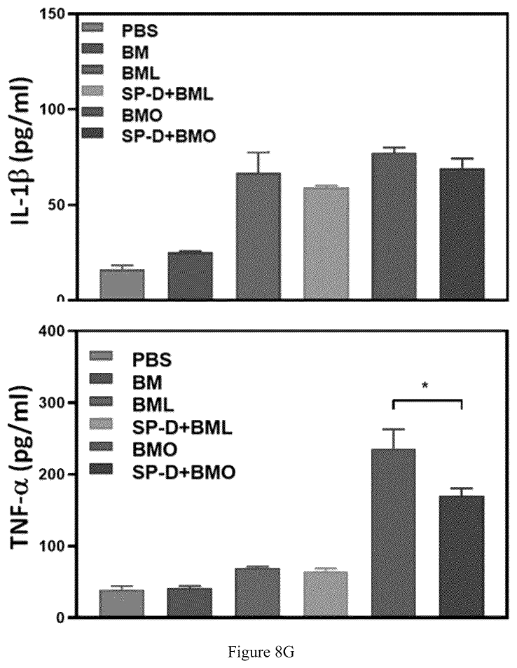

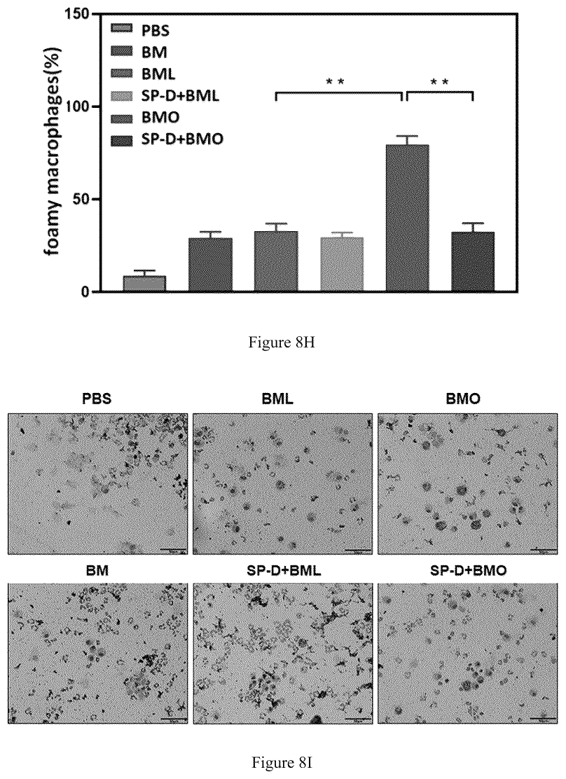

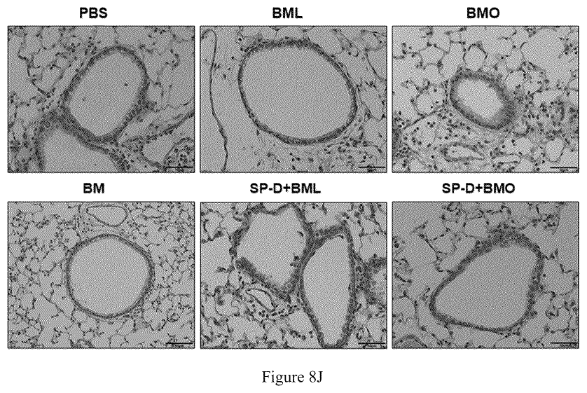

The adoptive transfer experiments were conducted to investigate whether FMs induced inflammation in the lung. Naïve mice were adoptively transferred medium-, LDL-, and oxLDL-treated BMDMs on Day 0. The mice were then intratracheally administered PBS or LPS on Day 2 (FIG. 8A). OxLDL-treated BMDMs significantly induced cell infiltration and IL-1β and TNF-α production in the lungs of LPS-treated mice but not PBS-treated mice (FIGS. 8B and 8C). In LPS-treated mice, oxLDL-treated BMDMs resulted in higher small airway thickness than that in control mice (FIG. 8D). After confirming that oxLDL-treated BMDMs induced more severe inflammation in LPS-treated mice, we examined whether rfhSP-D-pretreated FMs reduced inflammation in the LPS treatment model (FIG. 8E). Mice that received SP-D plus oxLDL-treated BMDMs had significantly reduced total cell numbers (FIG. 8F), levels of TNF-α (FIG. 8G), and percentages of total foamy cells (FIG. 8H), the images of the foamy cells (FIG. 8I), showing the alleviation of the thickened trachea (FIG. 8J) in the lungs compared to those in the oxLDL-treated BMDM groups.

SP-D Inhibited FM-Induced Impaired Lung Function and Airway Inflammation in Mice with Ozone-Induced COPD

Next, the effect of exogenous FM administration was examined on an ozone-induced mouse model of COPD. BMDMs were treated with the medium (BM), LDL (BML), and oxLDL (BMO) for 1 day, and then intratracheal adoptive transfer with BMDMs was performed into the lungs of ozone-exposed mice, as shown in the protocol (FIG. 7F). Impaired lung function was further exacerbated in ozone-exposed mice in the BMO (O-BMO) group (FIG. 7G). These mice had higher mucus accumulation in their airways compared to those in the other treatment groups (FIG. 7H). However, no differences were found in inflammatory cell infiltration among the ozone-exposed groups (FIG. 7I). These data suggest that FMs enhance ozone-induced airway inflammation in the lung.

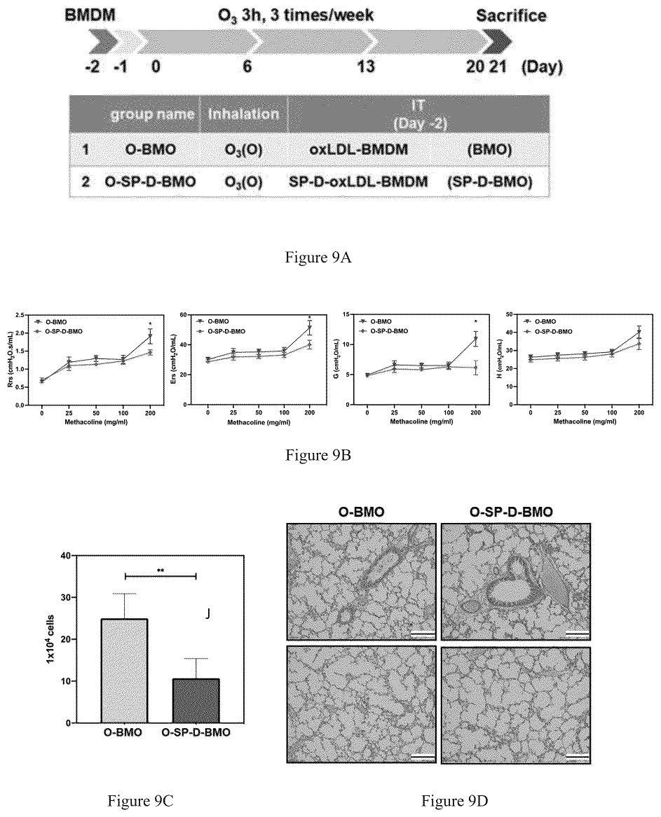

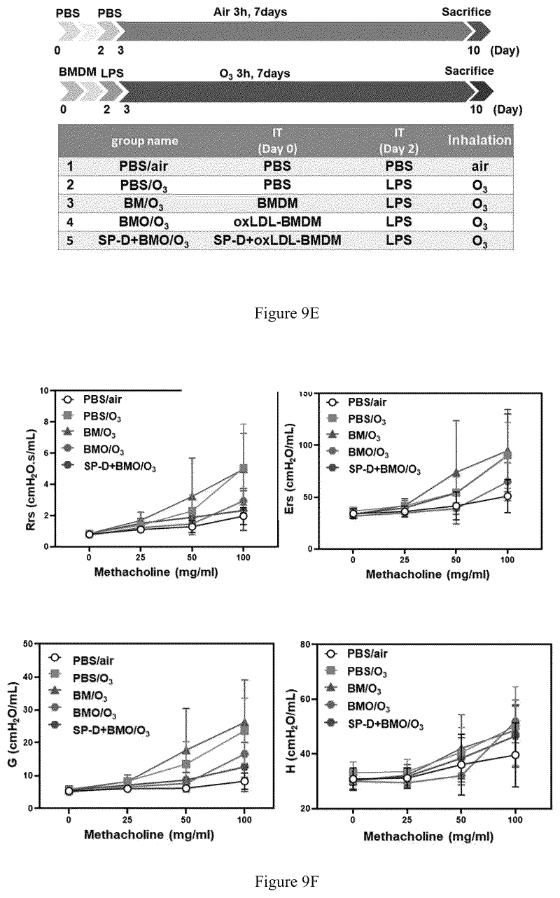

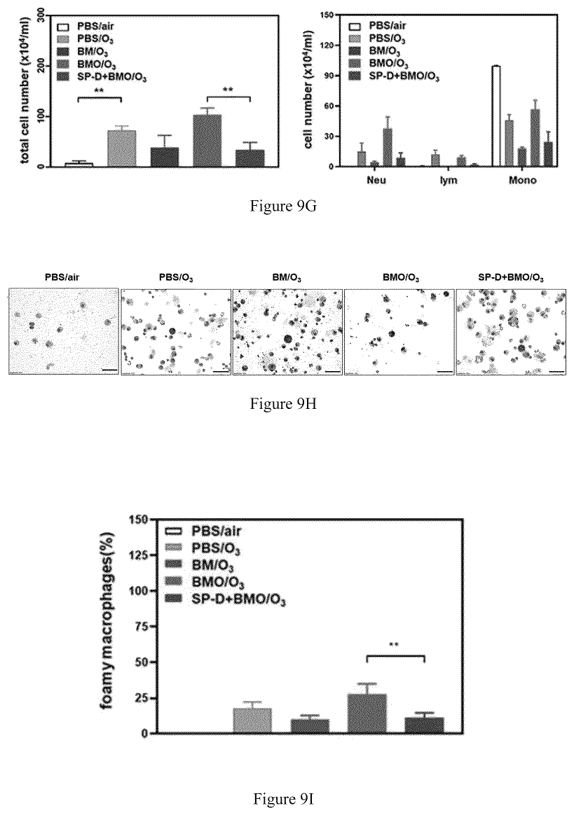

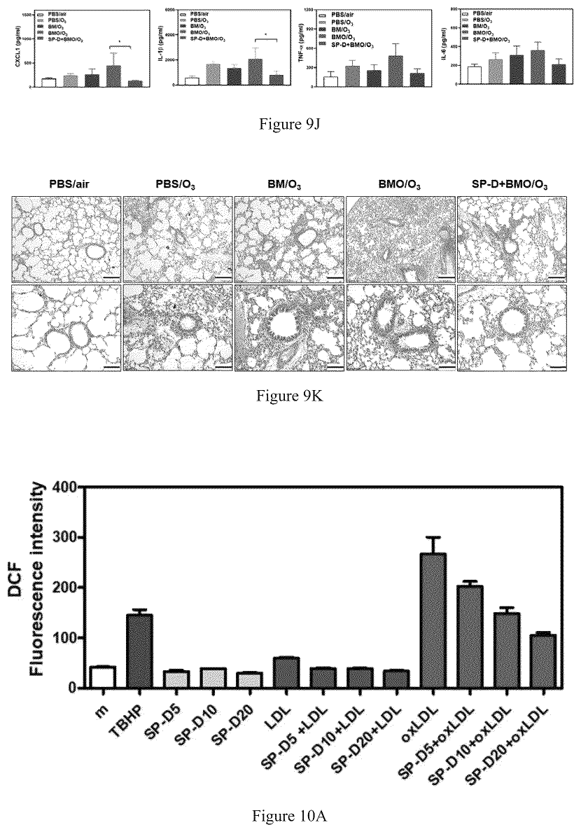

To investigate whether SP-D inhibited FM-induced lung inflammation in ozone-exposed COPD mice, BMDMs were first pretreated with SP-D and stimulated with oxLDL (SP-D-BMO) and then adoptively transferred into ozone-exposed mice (FIG. 9A). SP-D-BMO alle-viated the impaired lung function (FIG. 9B) and reduced cell infiltration (FIG. 9C) and mucus production in the airways (FIG. 9D) of recipient mice compared to those that received only oxLDL-treated BMDMs (BMO). To mimic the severe acute exacerbation of COPD in mice during bacterial infections, we first administered low-dose (10 μg/ml) LPS intratracheally and then subjected the mice to chronic ozone exposure for 7 days (FIG. 9E). Although mice treated with SP-D-BMO exhibited no differences in respiratory mechanics compared to those in the other groups (FIG. 9F), SP-D-BMO significantly decreased the total infiltrated cells (FIG. 9G) and the percentage of FMs (FIGS. 9H and 9I) and reduced the production of CXCL1, IL-1β, TNF-α and IL-6 (FIG. 9J) in the BALF of recipient mice compared to the mice that received only oxLDL-treated BMDMs. Mice treated with SP-D-BMO showed significantly reduced inflammatory cell infiltration in airways and alveoli (FIG. 9K). These data suggest that SP-D may improve impaired lung function and inflammation in COPD by reversing the pathogenesis of FMs.

SP-D Modified Gene Expression in oxLDL-Treated BMDMs

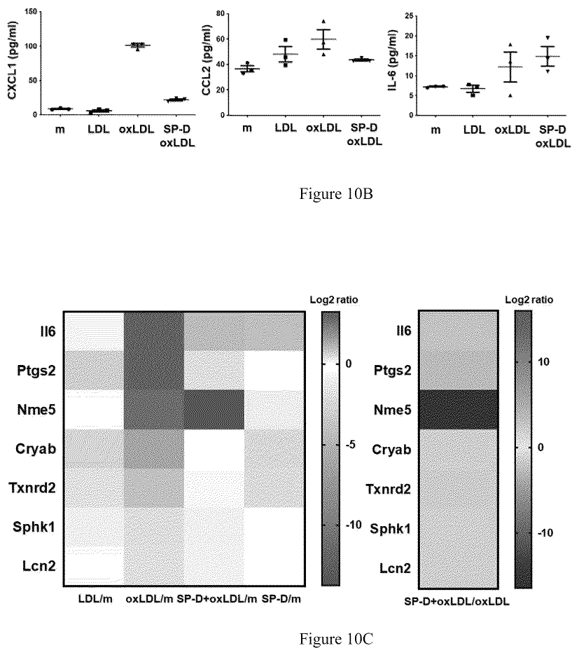

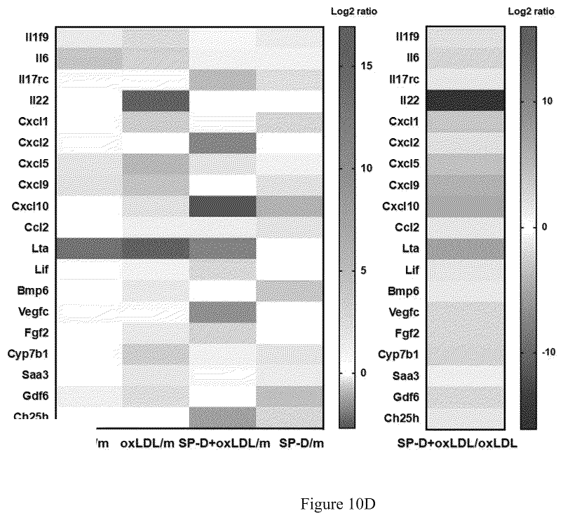

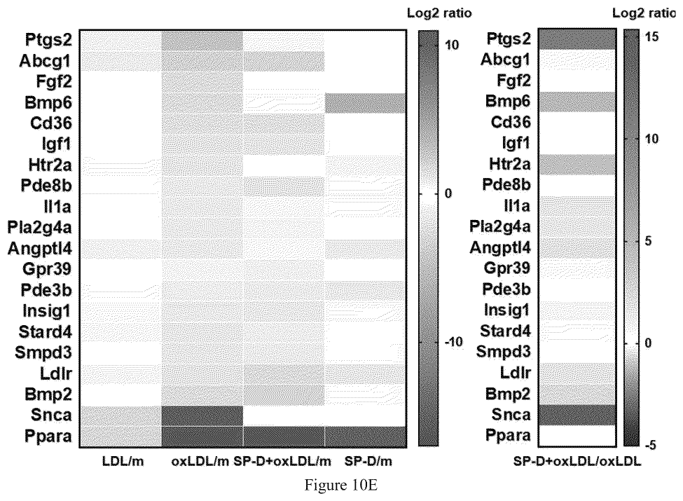

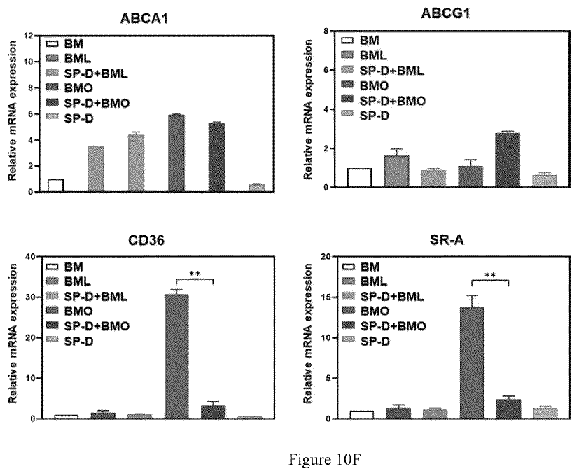

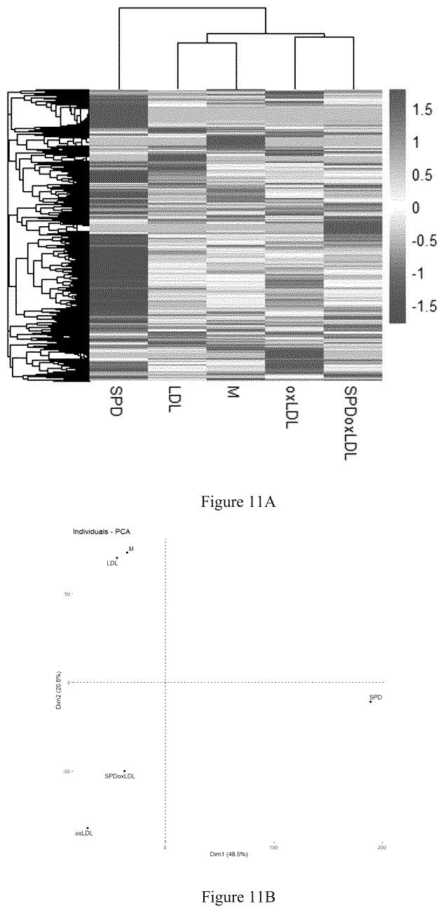

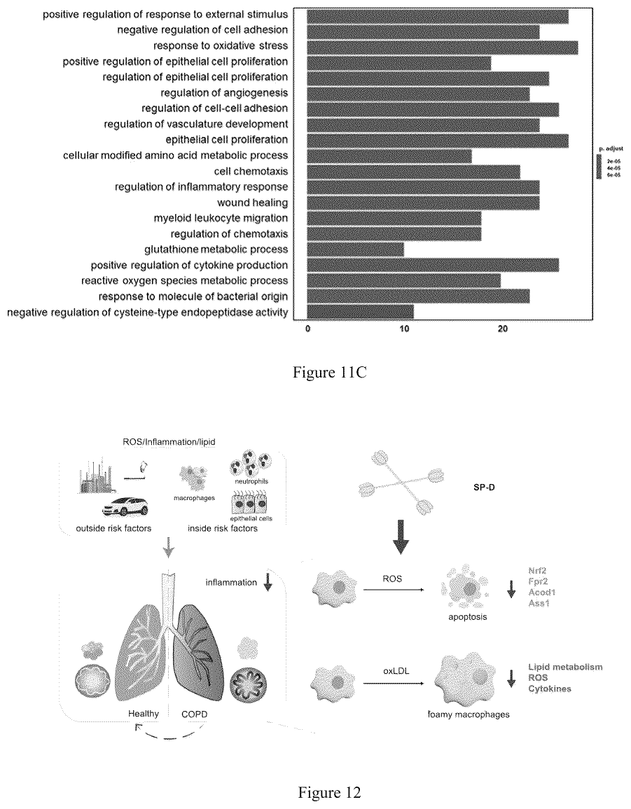

To investigate how SP-D regulates the inflammatory function and gene expression of FMs, we analyzed the levels of ROS, cytokines, and RNA expression in LDL-, oxidized LDL-, oxidized LDL plus SP-D-, and SP-D-treated BMDMs. Oxidized LDL-treated BMDMs had higher levels of ROS than medium- or LDL-treated BMDMs. SP-D reduced the ROS production of oxLDL-treated BMDMs in a dose-dependent manner (FIG. 10A). Moreover, oxLDL-treated BMDMs had increased expression of CXCL1, CCL2, and IL-6, whereas pretreatment with SP-D reduced the expression of CXCL1 and CCL2 in oxLDL-treated BMDMs (FIG. 10B). The RNA-seq and PCA results showed that the gene expression profile can be clearly divided into three clusters: medium (M) and LDL treatment (LDL); oxLDL treatment (oxLDL) and SP-D plus oxLDL treatment (SPD+oxLDL); and SP-D treatment (SP-D) (FIGS. 11A, 11B). OxLDL was shown to regulate the expression of genes involved in several biological processes, such as the response to oxidative stress, inflammation, and toxic substances (FIG. 11C). It was found that oxLDL increased the level of oxidative stress-(FIG. 10C) and cytokine activity (FIG. 10D)-related genes, while SP-D pretreatment reversed this oxLDL-induced upregulation. SP-D pretreatment also reduced the expression of several genes involved in lipid metabolism and significantly increased the expression of alpha-synuclein (SNCA), which mediates lipid transport in the brain (FIG. 10E). Moreover, SP-D pretreatment significantly decreased the RNA expression of the lipid uptake receptors CD36 and SR-A compared to that in oxLDL-treated BMDMs (FIG. 10F). These findings suggested that SP-D could reduce FM formation by mediating lipid metabolism.

### CONCLUSION

Various mechanisms were explored by which SP-D can treat ozone- and CS-induced airway inflammation and emphysematous destruction using experimental models of COPD. It was found increased levels of SP-D in the BALF and sera of ozone- and CS-exposed mice. SP-D-knockout mice were susceptible to the accumulation of lipid-laden FMs and macrophage-rich airway inflammation caused by ozone. Importantly, exogenous rfhSP-D treatment prevented the formation of oxLDL-induced FMs in vitro and reversed the airway inflammation and emphysematous changes caused by oxidative stress in vivo. In the present invention, it was concluded that SP-D upregulated genes in BMDMs that counter oxidative stress and lipid metabolism perturbations induced by CS and oxidized cholesterols, showing the crucial roles of SP-D in the immunometabolism of AMs and the restoration of lipid homeostasis disrupted by ozone and CS exposure in COPD.

It was clearly showed that direct administration of oxLDL-induced FMs into the trachea induced airway inflammation in recipient mice (see FIG. 7) and exacerbated emphysematous lung changes induced by ozone exposure (FIG. 7) and LPS stimulation (FIG. 8). The administration of recombinant full-length or a fragment of human SP-D could prevent or alleviate airway inflammation and emphysematous alveolar destruction in recipient mice during chronic ozone or CS exposure (FIG. 4).

It was evidenced that SPD in COPD plays a protective role in view that SP-D could prevent oxLDL-induced FM formation in vitro (see FIG. 7E) and that pretreatment of these FMs with SP-D could reverse the FM-induced airway inflammation and emphysematous changes in ozone-exposed recipient mice (FIG. 9).

It is also ascertained that SP-D inhibited ROS production- and the expression of lipid metabolism-related genes (see FIG. 10). These effects were enhanced by oxLDL stimulation in BMDMs. Moreover, SP-D inhibited the RNA expression of the oxLDL-lipid uptake receptors CD36 and SR-A (see FIG. 10F), which may improve the reverse cholesterol transport pathway through ABCA1 and ABCG1 upregulation and increase the mobilization of cellular cholesterol to HDL particles in the extracellular space.

In summary, SP-D deficiency exacerbated airway inflammation, lipid-laden macrophage accumulation, and emphysematous alveolar destruction in ozone- and CS-exposed lungs. It was demonstrated in the present invention that local instillation of rfhSP-D, a recombinant fragment of human surfactant protein D, alleviated oxidative stress and CS-induced airway inflammation, decreased FM formation, and caused emphysematous changes in recipient mice. The biological benefits of SP-D in ozone and CS-induced COPD may be due to the inhibitory effects of SP-D on ROS production by dysfunctional AMs and the restoration of lipid metabolism and cellular machinery associated with reverse lipid transport in lipid-laden FMs (FIG. 12). It is concluded that SP-D plays a critical and protective role in the pathogenesis of ozone- and CS-induced COPD. Accordingly, the present invention provides a novel approach for treating COPD using SP-D.

While the present invention has been disclosed by way preferred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

### REFERENCE

1. Barnes P J, Burney P G, Silverman E K, Celli B R, Vestbo J, Wedzicha J A, et al. Chronic obstructive pulmonary disease. Nat Rev Dis Prim. 2015; 1:15076.
2. Cheng S L, Chan M C, Wang C C, Lin C H, Wang H C, Hsu J Y, et al. COPD in Taiwan: a national epidemiology survey. Int J Chron Obstruct Pulmon Dis. 2015; 10:2459-67.
3. Sullivan J, Pravosud V, Mannino D M, Siegel K, Choate R, Sullivan T. National and state estimates of COPD morbidity and mortality-United States, 2014-2015. Chronic Obstr Pulm Dis. 2018; 5:324-33.
4. Mittal M, Siddiqui M R, Tran K, Reddy S P, Malik A B. Reactive oxygen species in inflammation and tissue injury. Antioxid Redox Signal. 2014; 20:1126-67.
5. Cañadas O, Olmeda B, Alonso A, Pérez-Gil J. Lipid-protein and protein-protein interactions in the pulmonary surfactant system and their role in lung homeostasis. Int J Mol Sci. 2020; 21:3708.
6. Knudsen L, Ochs M, Mackay R, Townsend P, Deb R, Mühlfeld C, et al. Truncated recombinant human SP-D attenuates emphysema and type II cell changes in SPD deficient mice. Respir Res. 2007; 8:70.
7. Wright J R. Immunoregulatory functions of surfactant proteins. Nat Rev Immunol. 2005; 5:58-68.
8. Vandivier R W, Ogden C A, Fadok V A, Hoffmann P R, Brown K K, Botto M, et al. Role of surfactant proteins A, D, and C1q in the clearance of apoptotic cells in vivo and in vitro: calreticulin and CD91 as a common collectin receptor complex. J Immunol. 2002; 169:3978-86.
9. Knudsen L, Atochina-Vasserman E N, Massa C B, Birkelbach B, Guo C J, Scott P, et al. The role of inducible nitric oxide synthase for interstitial remodeling of alveolar septa in surfactant protein D-deficient mice. Am J Physiol Lung Cell Mol Physiol. 2015; 309: L959-69.
10. Korfhagen T R, Sheftelyevich V, Burhans M S, Bruno M D, Ross G F, Wert S E, et al. Surfactant protein-D regulates surfactant phospholipid homeostasis in vivo. J Biol Chem. 1998; 273:28438-43.
11. Wert S E, Yoshida M, LeVine A M, Ikegami M, Jones T, Ross G F, et al. Increased metalloproteinase activity, oxidant production, and emphysema in surfactant protein D gene-inactivated mice. Proc Natl Acad Sci USA. 2000; 97:5972-7.
12. Levitan I, Volkov S, Subbaiah P V. Oxidized LDL: diversity, patterns of recognition, and pathophysiology. Antioxid Redox Signal. 2010; 13:39-75.
13. Hirama N, Shibata Y, Otake K, Machiya J, Wada T, Inoue S, et al. Increased surfactant protein-D and foamy macrophages in smoking-induced mouse emphysema. Respirology. 2007; 12:191-201.
14. Lian X, Yan C, Yang L, Xu Y, Du H. Lysosomal acid lipase deficiency causes respiratory inflammation and destruction in the lung. Am J Physiol Lung Cell Mol Physiol. 2004; 286: L801-7.
15. Nandy D, Sharma N, Senapati S. Systematic review and meta-analysis confirms significant contribution of surfactant protein D in chronic obstructive pulmonary disease. Front Genet. 2019; 10:339.
16. Ju C R, Liu W, Chen R C. Serum surfactant protein D: biomarker of chronic obstructive pulmonary disease. Dis Markers. 2012; 32:281-7.
17. Wang H, Li F, Huang H, Wu F, Chen L, Zhang D, et al. Serum surfactant protein D is a potential biomarker for chronic obstructive pulmonary disease: a systematic review and meta-analysis. Clin Lab. 2019; 65.
18. Obeidat M, Li X, Burgess S, Zhou G, Fishbane N, Hansel N N, et al. Surfactant protein D is a causal risk factor for COPD: results of Mendelian randomisation. Eur Respir J. 2017; 50:1700657.
19. Ou C Y, Chen C Z, Hsiue T R, Lin S H, Wang J Y. Genetic variants of pulmonary SP-D predict disease outcome of COPD in a Chinese population. Respirology. 2015; 20:296-303.
20. Zhu H, Jia Z, Zhang L, Yamamoto M, Misra H P, Trush M A, et al. Antioxidants and phase 2 enzymes in macrophages: regulation by Nrf2 signaling and protection against oxidative and electrophilic stress. Exp Biol Med. 2008; 233:463-74.
21. Kotlyarov S, Kotlyarova A. Molecular mechanisms of lipid metabolism disorders in infectious exacerbations of chronic obstructive pulmonary disease. Int J Mol Sci. 2021; 22:7634.
22. Azimzadeh Jamalkandi S, Mirzaie M, Jafari M, Mehrani H, Shariati P, Khodabandeh M. Signaling network of lipids as a comprehensive scaffold for omics data integration in sputum of COPD patients. Biochim Biophys Acta. 2015; 1851:1383-93.
23. Ko F W, Lau C Y, Leung T F, Wong G W, Lam C W, Hui D S. Exhaled breath condensate levels of 8-isoprostane, growth related oncogene alpha and monocyte chemoattractant protein-1 in patients with chronic obstructive pulmonary disease. Respir Med. 2006; 100:630-8.
24. Bartoli M L, Novelli F, Costa F, Malagrinò L, Melosini L, Bacci E, et al. Malondialdehyde in exhaled breath condensate as a marker of oxidative stress in different pulmonary diseases. Mediators Inflamm. 2011; 2011:891752.
25. Montuschi P, Collins J V, Ciabattoni G, Lazzeri N, Corradi M, Kharitonov S A, et al. Exhaled 8-isoprostane as an in vivo biomarker of lung oxidative stress in patients with COPD and healthy smokers. Am J Respir Crit Care Med. 2000; 162:1175-7.
26. Miró O, Alonso J R, Jarreta D, Casademont J, Urbano-Márquez A, Cardellach F. Smoking disturbs mitochondrial respiratory chain function and enhances lipid peroxidation on human circulating lymphocytes. Carcinogenesis. 1999; 20:1331-6.
27. Ween M P, White J B, Tran H B, Mukaro V, Jones C, Macowan M, et al. The role of oxidised self-lipids and alveolar macrophage CD1b expression in COPD. Sci Rep. 2021; 11:4106.
28. Fessler M B. A new frontier in immunometabolism. Cholesterol in Lung health and disease. Ann Am Thorac Soc. 2017; 14: S399-405.
29. Morissette M C, Shen P, Thayaparan D, Stämpfli M R. Disruption of pulmonary lipid homeostasis drives cigarette smoke-induced lung inflammation in mice. Eur Respir J. 2015; 46:1451-60.
30. Fujii W, Kapellos T S, Baßler K, Händler K, Holsten L, Knoll R, et al. Alveolar macrophage transcriptomic profiling in COPD shows major lipid metabolism changes. ERJ Open Res. 2021; 7:00915-2020.
31. Baßler K, Fujii W, Kapellos T S, Horne A, Reiz B, Dudkin E, et al. Alterations of multiple alveolar macrophage states in chronic obstructive pulmonary disease. bioRxiv. 2020.
32. Fisher J H, Sheftelyevich V, Ho Y S, Fligiel S, McCormack F X, Korfhagen T R, et al. Pulmonary-specific expression of SP-D corrects pulmonary lipid accumulation in SP-D gene-targeted mice. Am J Physiol Lung Cell Mol Physiol. 2000; 278: L365-73.
33. Pilecki B, Wulf-Johansson H, Støttrup C, Jørgensen P T, Djiadeu P, Nexøe A B, et al. Surfactant protein D deficiency aggravates cigarette smoke-induced lung inflammation by upregulation of ceramide synthesis. Front Immunol. 2018; 9:3013.
34. Lara-Guzman O J, Gil-Izquierdo Á, Medina S, Osorio E, Alvarez-Quintero R, Zuluaga N, et al. Oxidized LDL triggers changes in oxidative stress and inflammatory biomarkers in human macrophages. Redox Biol. 2018; 15:1-11.
35. Matsuura E, Hughes G R, Khamashta M A. Oxidation of LDL and its clinical implication. Autoimmun Rev. 2008; 7:558-66.
36. Malur A, Baker A D, Mccoy A J, Wells G, Barna B P, Kavuru M S, et al. Restoration of PPARgamma reverses lipid accumulation in alveolar macrophages of GM-CSF knockout mice. Am J Physiol Lung Cell Mol Physiol. 2011; 300: L73-80.
37. Hafiane A, Gasbarrino K, Daskalopoulou S S. The role of adiponectin in cholesterol efflux and HDL biogenesis and metabolism. Metabolism. 2019; 100:153953.
38. Alzoubi A, Ghazwi R, Alzoubi K, Alqudah M, Kheirallah K, Khabour O, et al. Vascular endothelial growth factor receptor inhibition enhances chronic obstructive pulmonary disease picture in mice exposed to waterpipe smoke. Folia Morphol. 2018; 77:447-55.
39. Dodagatta-Marri E, Qaseem A S, Karbani N, Tsolaki A G, Waters P, Madan T, et al. Purification of surfactant protein D (SP-D) from pooled amniotic fluid and bronchoalveolar lavage. Methods Mol Biol. 2014; 1100:273-90.
40. Hsieh M H, Beirag N, Murugaiah V, Chou Y C, Kuo W S, Kao H F, et al. Human surfactant protein D binds spike protein and acts as an entry inhibitor of SARSCoV-2 pseudotyped viral particles. Front Immunol. 2021; 12:641360.
41. Assouvie A, Daley-Bauer L P, Rousselet G. Growing murine bone marrow-derived macrophages. Methods Mol Biol. 2018; 1784:29-33.
42. Hirai T, Fukui Y, Motojima K. PPARalpha agonists positively and negatively regulate the expression of several nutrient/drug transporters in mouse small intestine. Biol Pharm Bull. 2007; 30:2185-90.
43. Kang J H, Ko H M, Han G D, Lee S Y, Moon J S, Kim M S, et al. Dual role of phosphatidylserine and its receptors in osteoclastogenesis. Cell Death Dis. 2020; 11:497.
44. Sekine S, Yao A, Hattori K, Sugawara S, Naguro I, Koike M, et al. The ablation of mitochondrial protein phosphatase Pgam5 confers resistance against metabolic stress. EBioMedicine. 2016; 5:82-92.
45. He X, Chen X, Wang L, Wang W, Liang Q, Yi L, et al. Metformin ameliorates Ox-LDL-induced foam cell formation in raw264.7 cells by promoting ABCG-1 mediated cholesterol efflux. Life Sci. 2019; 216:67-74.

What is claimed is:

1. A method for treating chronic obstructive pulmonary disease (COPD) in a subject, comprising administering to said subject a therapeutically effective amount of surfactant protein-D (SP-D).

2. The method of claim 1, wherein the SP-D is a native SP-D.

3. The method of claim 2, wherein the native SP-D consists of the amino acid sequence of SEQ ID NO: 1.

4. The method of claim 1, wherein the SP-D is a recombinant fragment of human SP-D (rfhSP-D).

5. The method of claim 4, wherein the rfhSP-D is the fragment consisting of the amino acid sequence of SEQ ID NO:2.


SEQUENCE LISTING (12 sequences)

SEQ ID NO: 1 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 2 - protein, 177 aa, Homo sapiens
GSPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 3 - DNA, 20 bp, synthetic construct
  source 1..20: mol_type = other DNA
gcagatcaagcatcccaact

SEQ ID NO: 4 - DNA, 22 bp, synthetic construct
  source 1..22: mol_type = other DNA
ccagagaatgtttcattgtcca

SEQ ID NO: 5 - DNA, 20 bp, synthetic construct
  source 1..20: mol_type = other DNA
gggtctgaactgccctacct

SEQ ID NO: 6 - DNA, 20 bp, synthetic construct
  source 1..20: mol_type = other DNA
tactcccctgatgccacttc

SEQ ID NO: 7 - DNA, 27 bp, synthetic construct
  source 1..27: mol_type = other DNA
ttgtacctatactgtggctaaatgaga

SEQ ID NO: 8 - DNA, 20 bp, synthetic construct
  source 1..20: mol_type = other DNA
tctaccatgccaaggagctt

SEQ ID NO: 9 - DNA, 18 bp, synthetic construct
  source 1..18: mol_type = other DNA
gcatcccttcctcacagc

SEQ ID NO: 10 - DNA, 18 bp, synthetic construct
  source 1..18: mol_type = other DNA
gcatcccttcctcacagc

SEQ ID NO: 11 - DNA, 22 bp, synthetic construct
  source 1..22: mol_type = other DNA
ggtcatccatgacaactttggc

SEQ ID NO: 12 - DNA, 22 bp, synthetic construct
  source 1..22: mol_type = other DNA
tggatgcagggatgatgttctg